(19) United States
(12) Patent Application Publication
(10) Pub. No.: US 2005/0069853 A1
(43) Pub. Date: Mar. 31, 2005

(54) Performance tracking systems and methods

(76) Inventors: William Randal Tyson, Jamestown, RI (US); John Augustus Wayt III, Jamestown, RI (US); Erwin R. Thomas, Duluth, GA (US); Kevin Edward Peterson, Roswell, GA (US); Keith Edward Kenyon, Westerly, RI (US); Robert Joseph Bouthillier, Lincoln, RI (US); Jorge Tomas, Wrentham, MA (US); Ming Q. Lac, East Greenwich, RI (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/950,864

(22) Filed: Sep. 27, 2004

Related U.S. Application Data
(60) Provisional application No. 60/506,271, filed on Sep. 26, 2003.

(51) Int. Cl. G09B 19/00; G09B 9/00; A63B 69/00; G09B 7/00; A61B 5/22

(52) U.S. Cl. 434/247; 434/350; 434/362; 434/219

(57) ABSTRACT

Embodiments of a performance tracking system and method are disclosed. One method embodiment, among others, includes receiving standardized physical performance test data over a network from a test site, the standardized physical performance test data corresponding to physical performance for a plurality of individuals, and processing the standardized physical performance test data to provide standardized data of physical performance among the plurality of individuals.

### CROSS-REFERENCE TO RELATED APPLICATION

This application claims the benefit of U.S. provisional application Ser. No. 60/506,271, filed Sep. 26, 2003, which is entirely incorporated herein by reference.

### TECHNICAL FIELD

The present invention is generally related to physical skills assessment and, more particularly, is related to physical performance test systems and methods.

### BACKGROUND

Physical skill tests may be used to evaluate athletic skills, occupational skills, among others. Most physical skill tests rely primarily on manual or semi-automated test procedures that use equipment and protocols that are subjective and fraught with deviation or systemic errors. Using such equipment and/or procedures may result in inconsistent evaluations, resulting in a lack of standardized, repeatable and reproducible data, especially when comparing such evaluations over multiple locations. The human component of many existing physical testing processes is also susceptible to overt or inadvertent assistance by the test evaluator.

### SUMMARY OF THE INVENTION

Preferred embodiments of a system and method are disclosed. One embodiment of a method, among other embodiments, includes receiving standardized physical performance test data over a network from a test site, the standardized physical performance test data corresponding to physical performance for a plurality of individuals, and processing the standardized physical performance test data to provide standardized data of physical performance among the plurality of individuals.

Other systems, methods, features, and advantages will be or become apparent to one with skill in the art upon examination of the following drawings and detailed description. It is intended that all such additional systems, methods, features, and advantages be included within this description and be within the scope of the disclosure.

### BRIEF DESCRIPTION OF THE DRAWINGS

Many aspects of embodiments of a system and method can be better understood with reference to the following drawings. The components in the drawings are not necessarily to scale, emphasis instead being placed upon clearly illustrating the principles of systems and methods. Moreover, in the drawings, like reference numerals designate corresponding parts throughout the several views.

FIG. 1 is a block diagram of a performance tracking system.

FIG. 2A is a schematic diagram depicting an embodiment of the performance tracking system shown in FIG. 1.

FIG. 2B is a block diagram of an embodiment of a central server that can implement performance tracking software of the performance tracking system shown in FIG. 1.

FIGS. 2C-2I are flow diagrams that depict method embodiments of the performance tracking software shown in FIG. 2B.

FIGS. 3A-3C are block diagrams that illustrate functionality provided by web-interface screens provided by the performance tracking software shown in FIG. 2B.

FIG. 4A is a schematic diagram that depicts an embodiment of a test station in front elevation view as shown in FIG. 2A.

FIG. 4B is a schematic diagram that depicts a side view of the test station shown in FIG. 4A.

FIG. 4C is a schematic diagram that depicts an embodiment of a controller in front elevation view used in the test station shown in FIGS. 4A-4B.

FIG. 4D is a schematic diagram that depicts a side view of the controller shown in FIG. 4C.

FIG. 4E is a flow diagram that depicts a method embodiment 400a of the test station module 400 shown in FIGS. 4A-4B.

FIG. 5 is a block diagram that illustrates functionality for a web-interface embodiment provided by the performance testing software for a server located at a performance lab as shown in FIG. 1.

FIG. 6 is a schematic diagram that depicts one embodiment of a performance lab as shown in FIG. 1.

FIGS. 7A-7H are schematic diagrams that illustrate exemplary test stations illustrated in FIG. 6.

### DETAILED DESCRIPTION OF THE PREFERRED EMBODIMENTS

Preferred embodiments of a performance tracking system and method (herein referred to simply as a performance tracking system) are disclosed. A performance tracking system include mechanisms for quantifying assessment of individuals' physical skills using networked automatic measuring devices, software, and/or hardware. In a performance tracking system, physical skills evaluations are assessed and recorded using secure proprietary networked testing and methodology, software and equipment in one or more authorized physical performance laboratories. The skills data is thereafter transmitted from a performance laboratory (herein, performance lab) over a network, and processed with performance tracking software to create a relational database that can be sorted by numerous criteria. The data can be automatically processed and compiled into a statistical numerical comparative score, or a distinct set of numerical values, in a secure computer database. For example, data may be inputted into a membership computer database which is configured with performance tracking software to compute and create a score or a set of numerical values that can be used for comparison purposes within defined parameters or standards.

A performance tracking system includes, but is not limited to, performance tracking software accessible on the World Wide Web (Internet) that allows for interaction of member participants and certified testing organizations. Performance tracking software and/or hardware is packaged into a protocol that is repeatable and reproducible in multiple authorized locations, which enables a member participant to be evaluated and compared on a quantitatively and statistically valid method.

A performance tracking system provides for the exchange of information between students or other individuals and academic and/or occupational institutions, providing methodologies and resources to quantify the assessment of physical prowess for comparison and improvement. A performance tracking system provides a method for a testing organization to generate useful comparative data and can provide an additional source of funding for athletic or occupational programs. A performance tracking system can be used to assess large and/or fine motor skills. A performance tracking system provides the ability to quantify the physical attributes of individuals, which is useful for personal development, admissions evaluations for college and high school athletic programs, as well as evaluations for occupational programs. A performance tracking system can be a resource to the institutions and the member participants (e.g., individual student athletes, candidates or potential candidates for various occupational positions, etc.) as the database can serve as a communications center for the exchange of data for both member participants and member institutions. These assessments can be used for indicators of potential success in occupations that demand physical skill (such as firemen, police etc.) and/or specific eye-hand coordination (such as dentist, pilot, laser surgeon etc.).

A performance tracking system can enable the development of programs to assist in the evaluation of physically challenged individuals. This program may incorporate performance tracking methodology as an outreach to provide opportunities for career placement for the physically challenged.

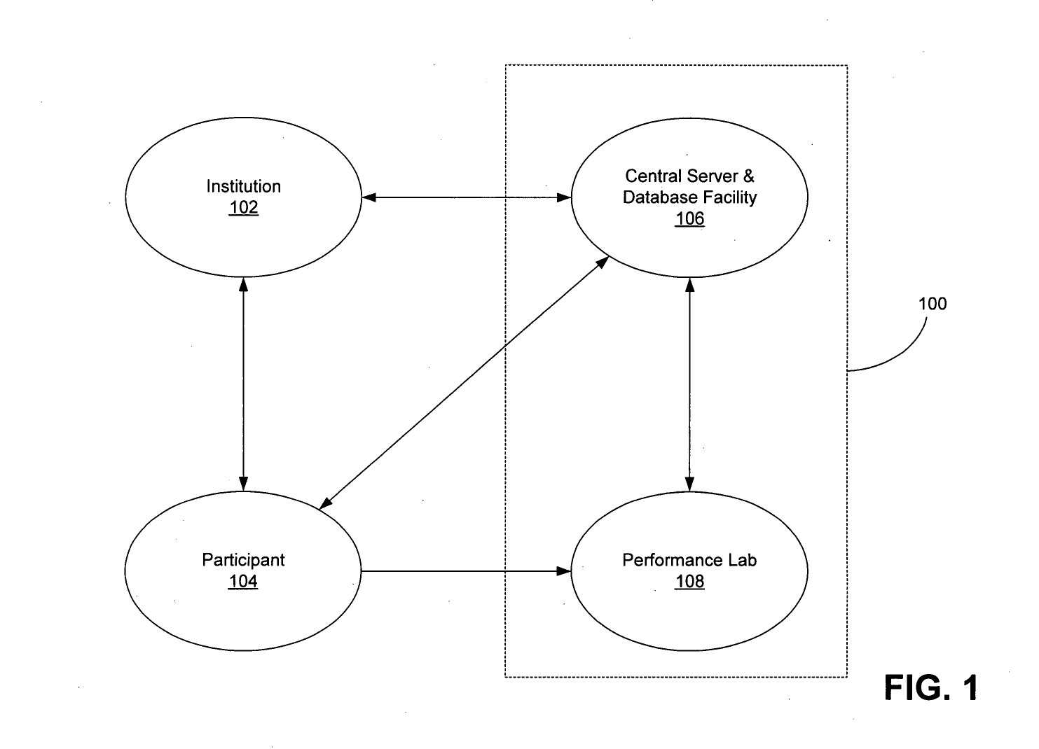

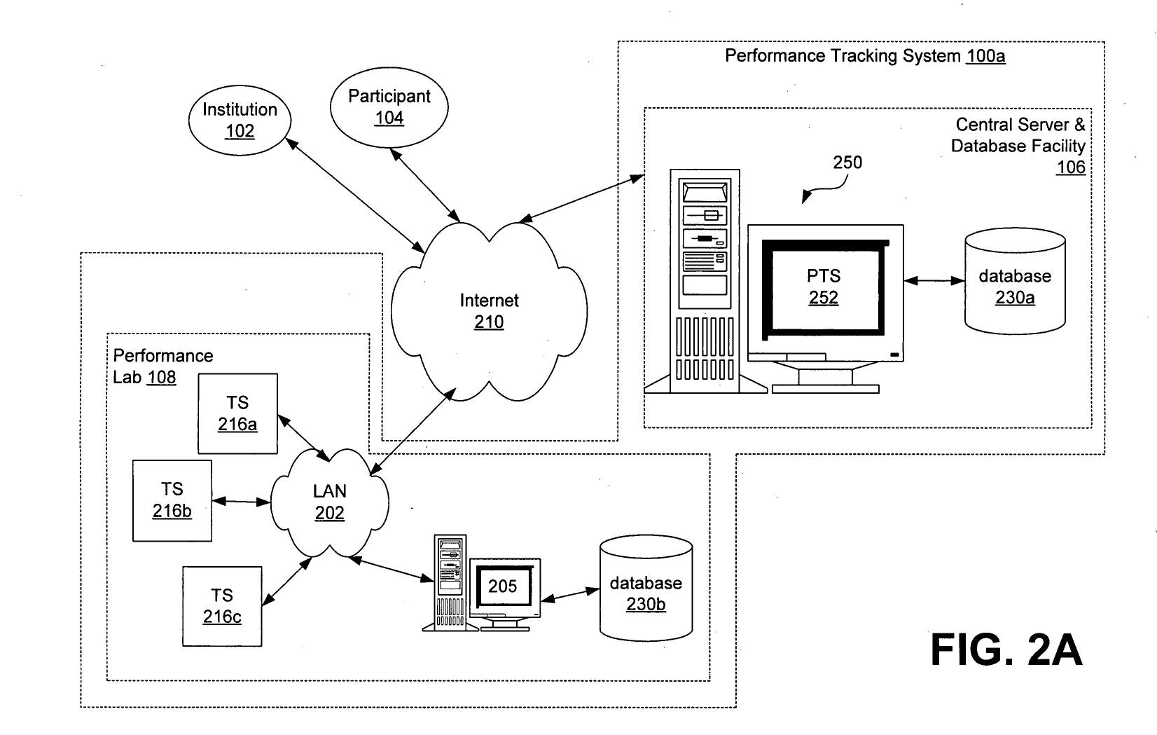

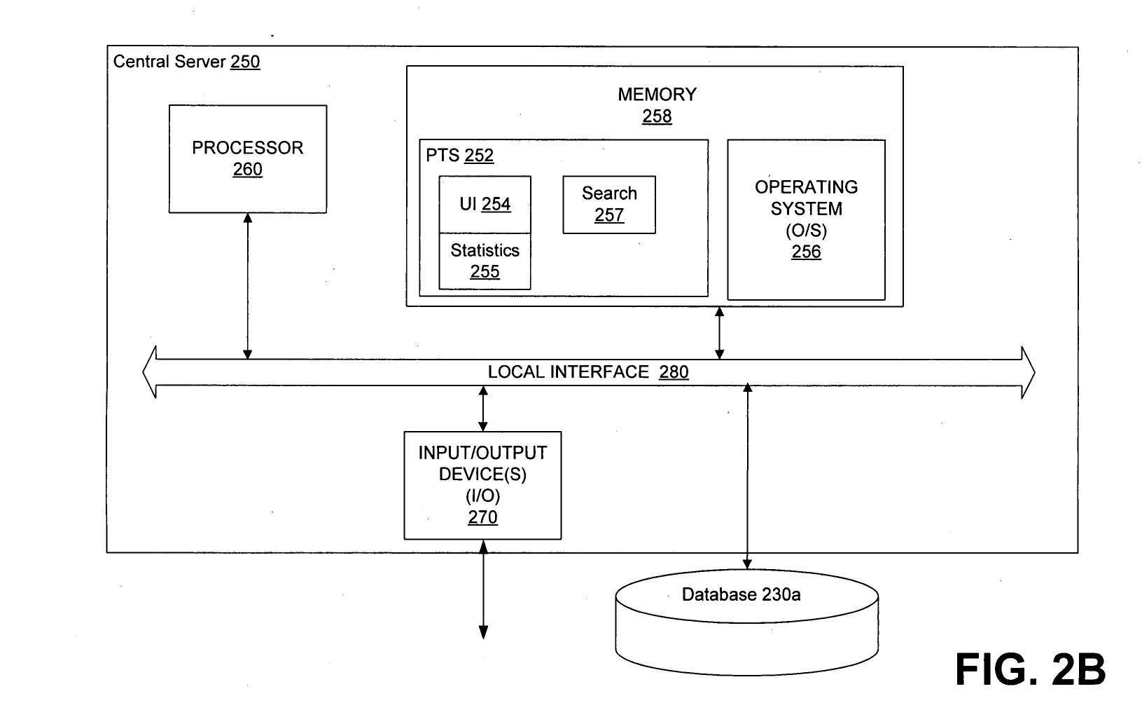

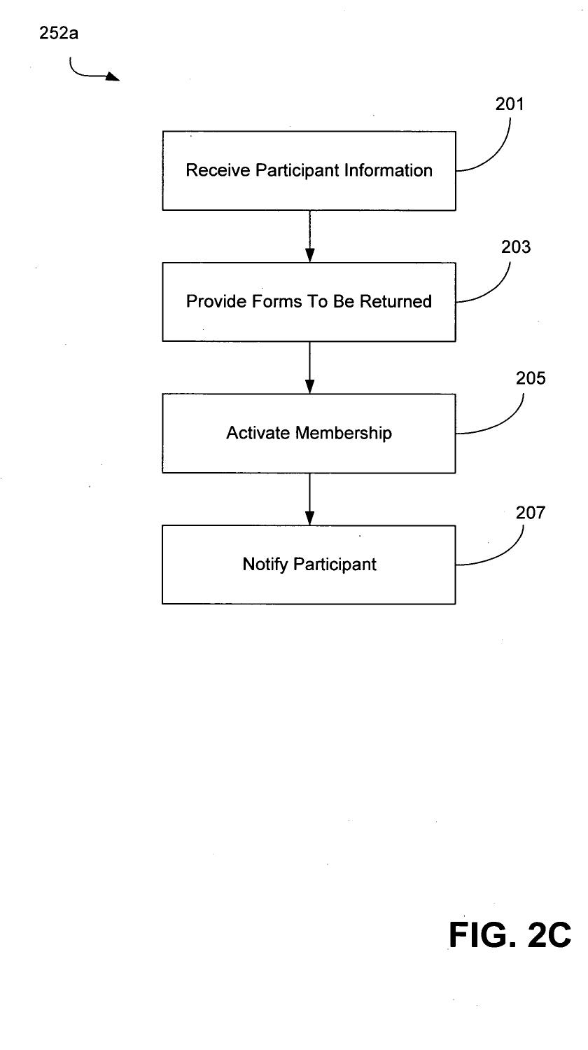

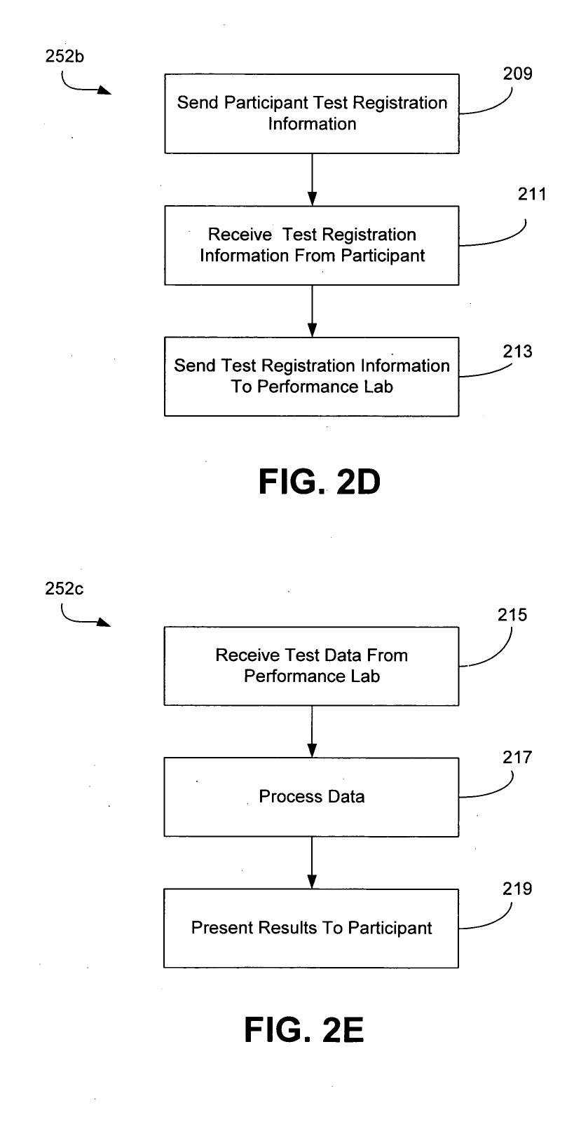

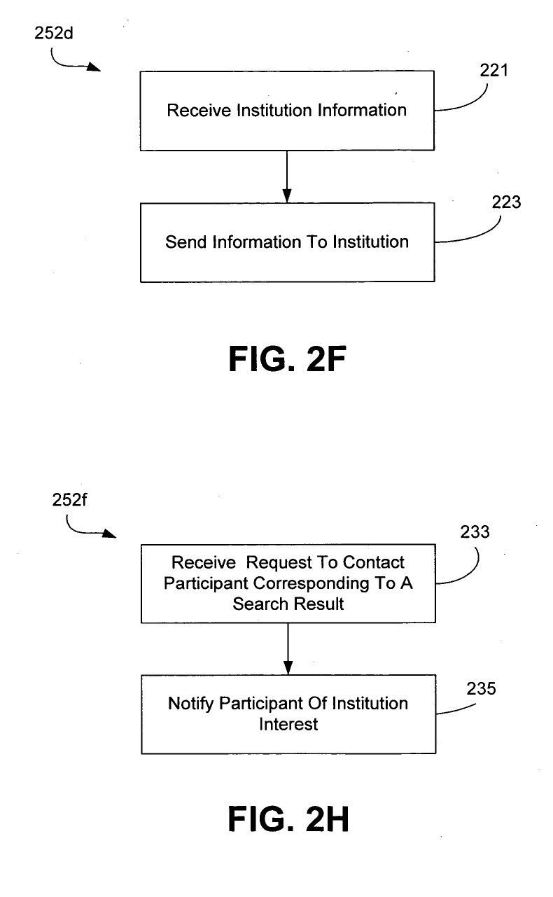

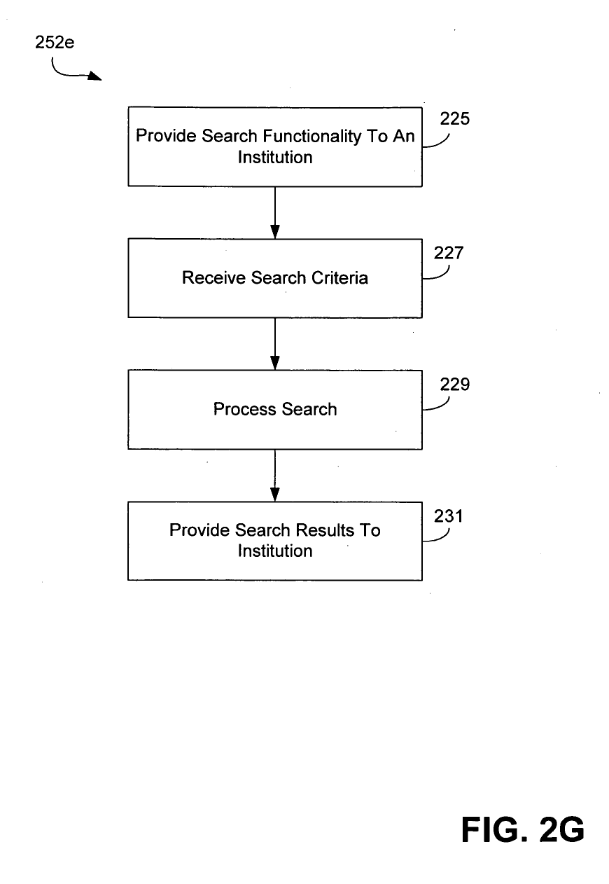

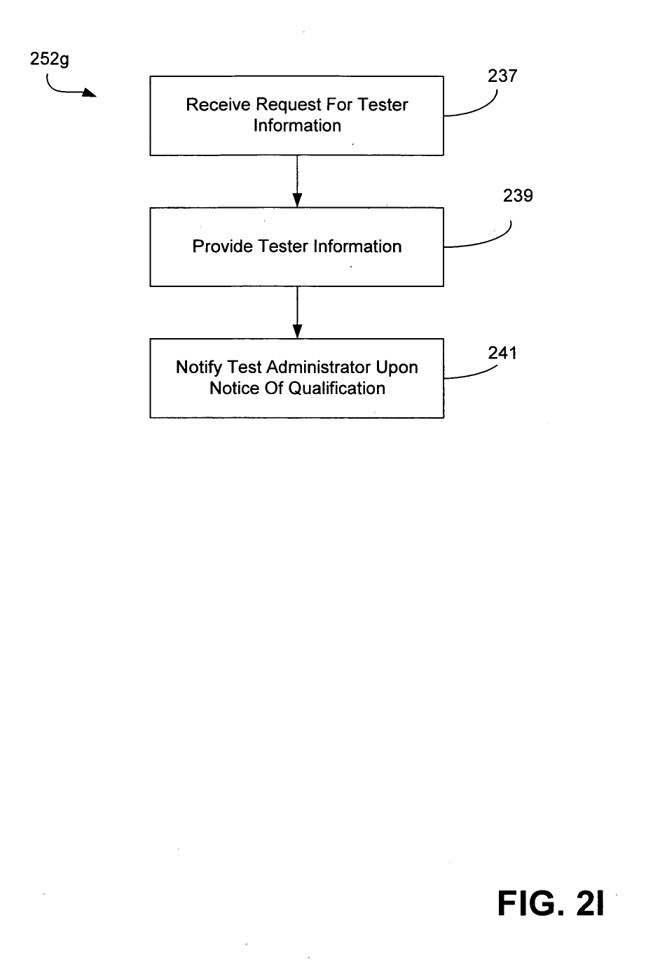

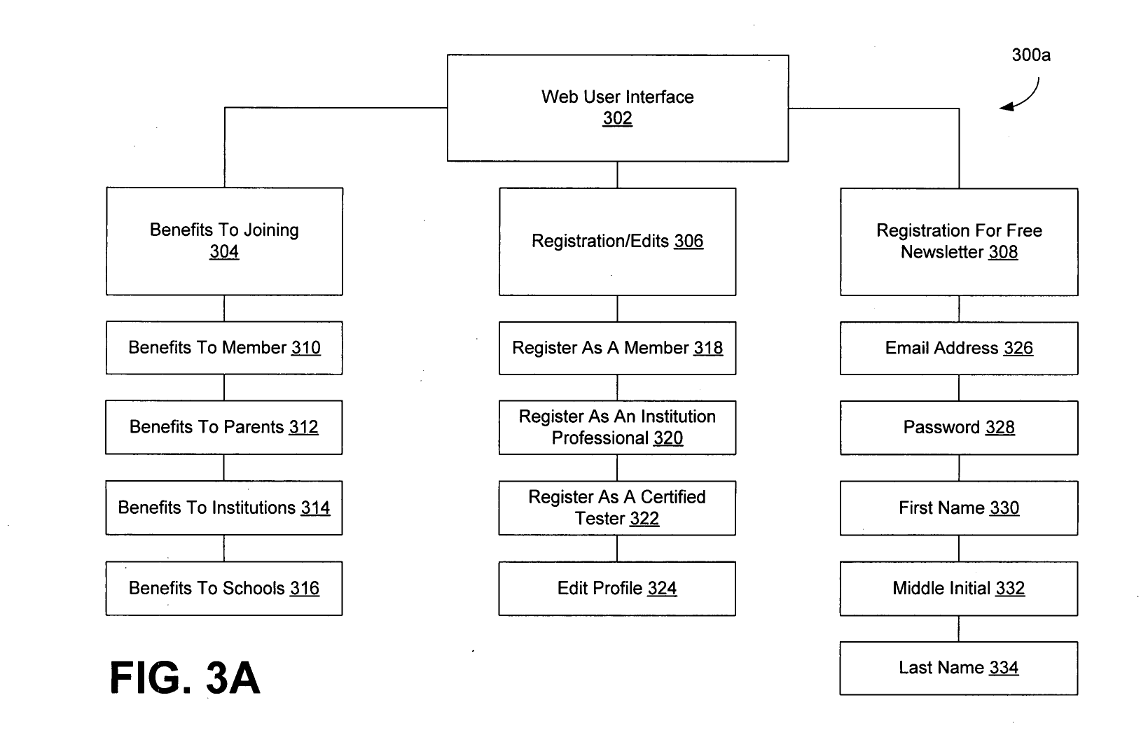

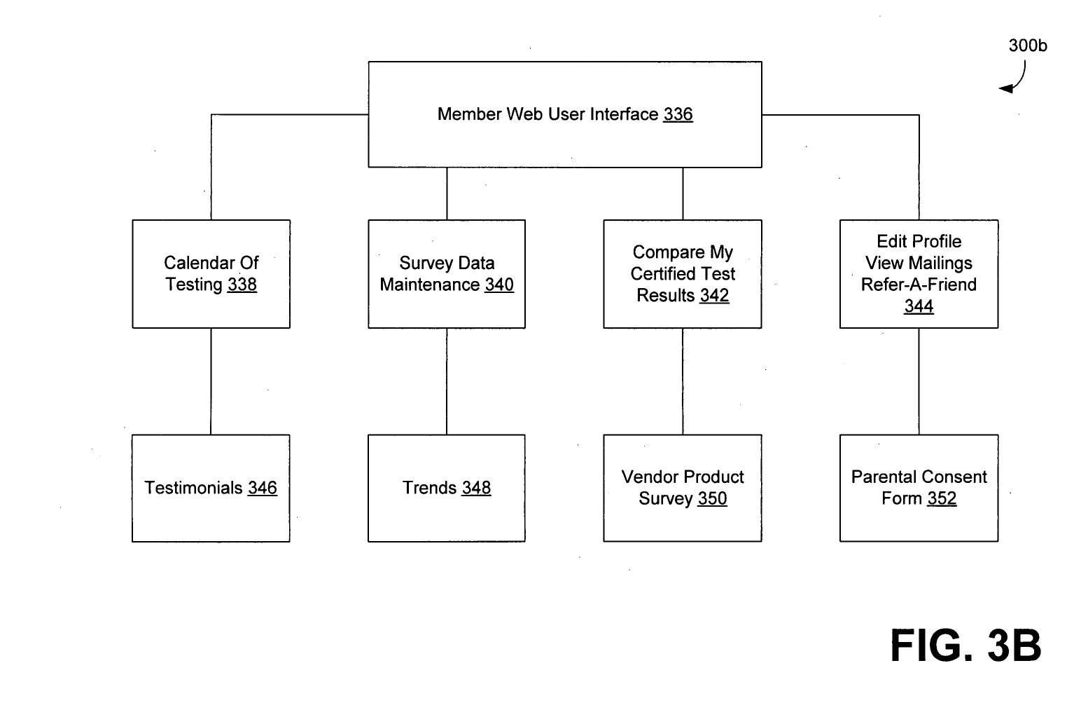

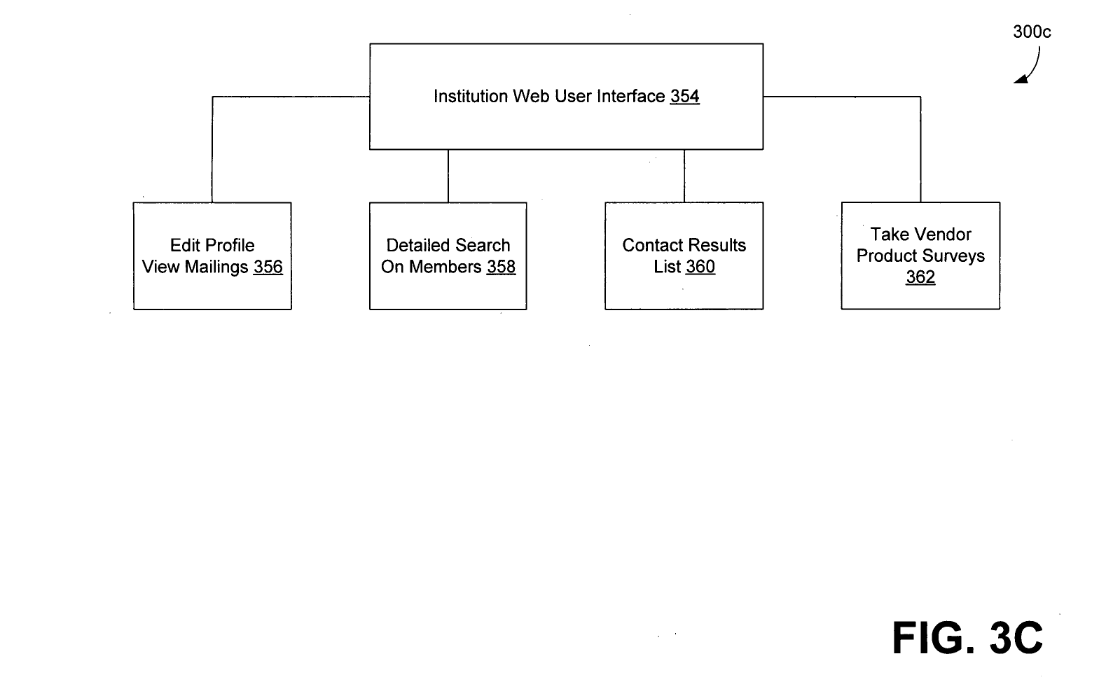

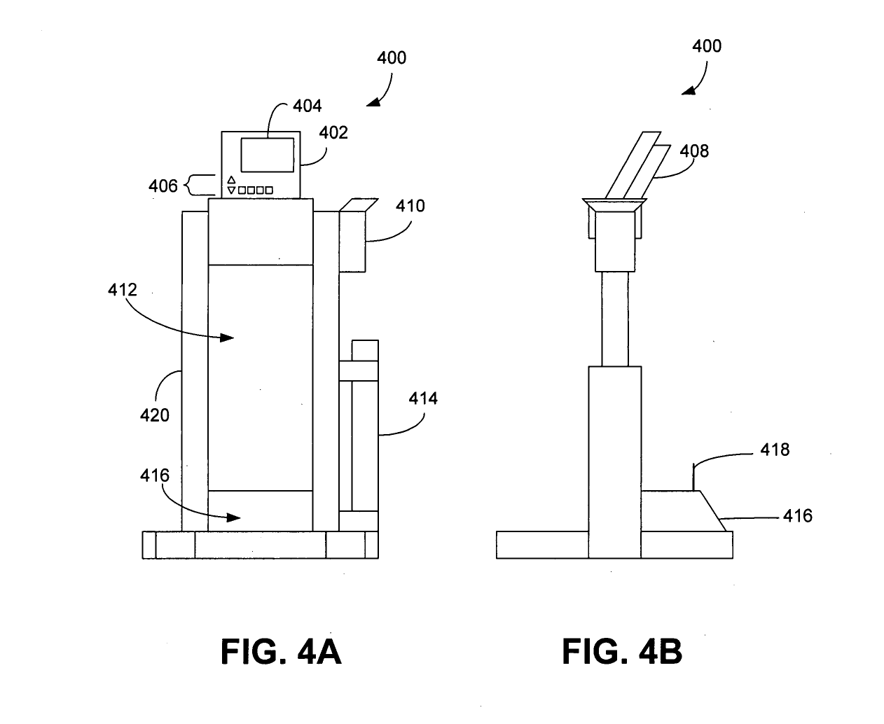

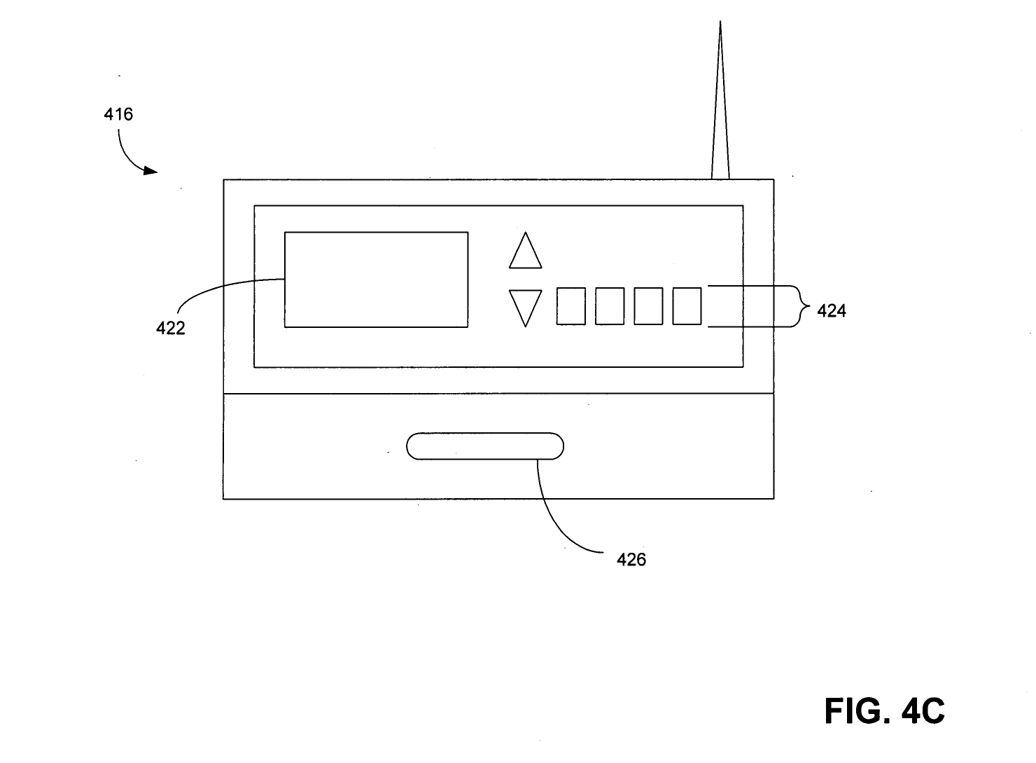

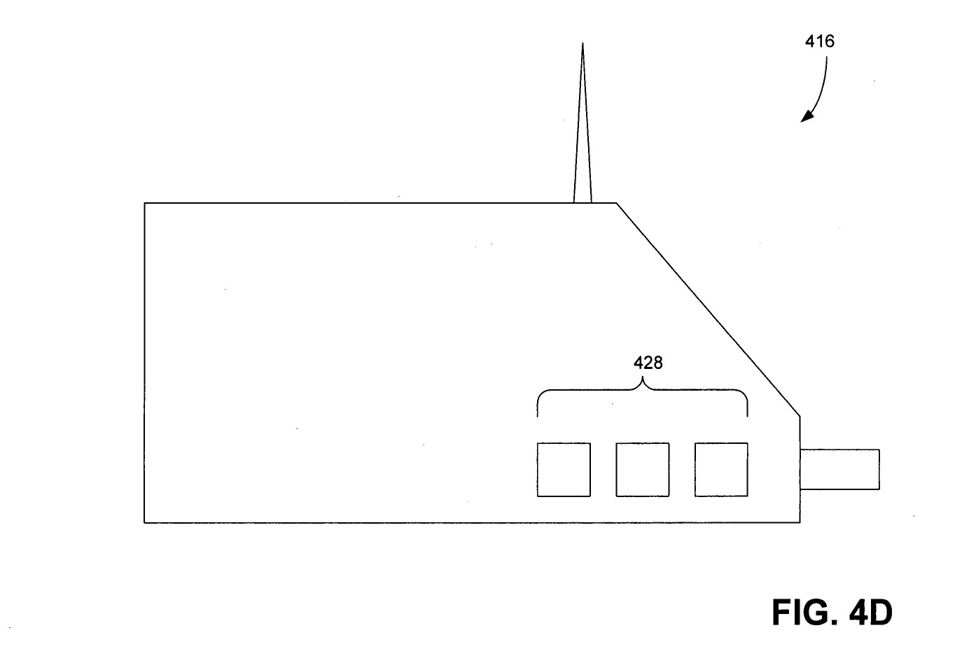

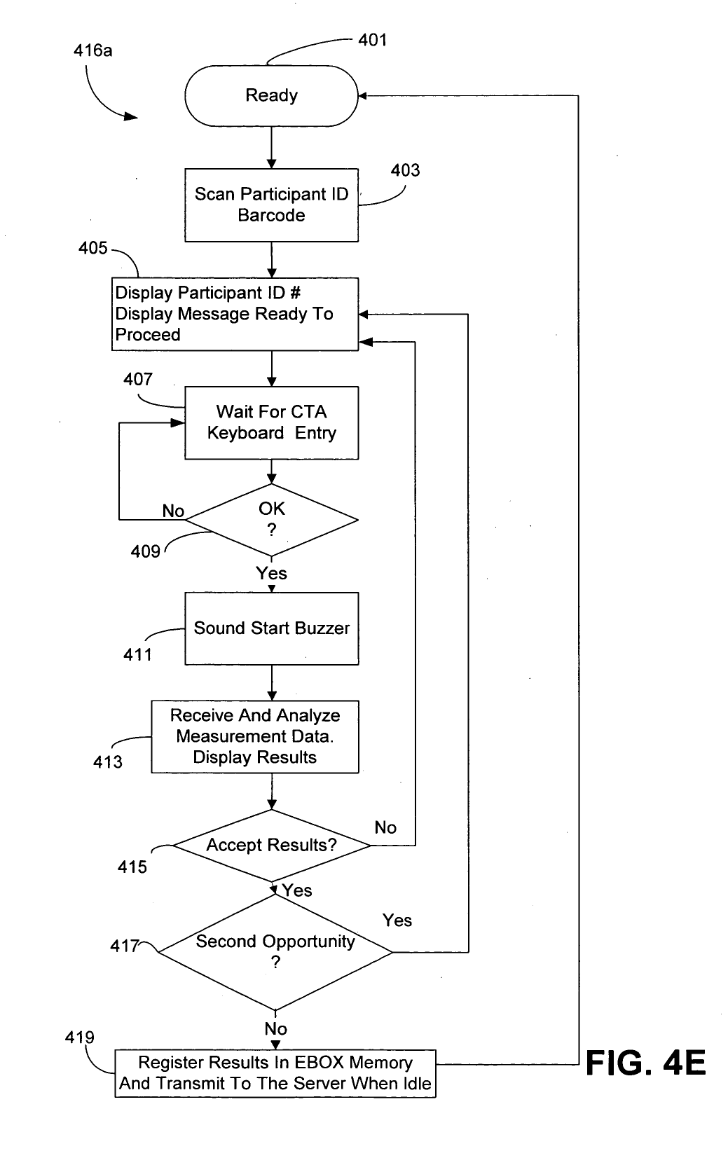

Preferred embodiments of a performance tracking system is described in association with FIG. 1, along with components and software methods as illustrated in FIGS. 2A-3C as implemented for athletic physical skills assesments. An example performance lab and components and associated methods are illustrated in FIGS. 4A-7H. It will be understood that the disclosed systems and methods also encompass in scope other physical skill assessment implementations, such as occupational skills testing, among others.

FIG. 1 illustrates an embodiment of a performance tracking system 100. The performance tracking system 100 includes a central server and database facility 106 and one or more performance labs 108. The performance tracking system 100 interacts with one or more member institutions 102 and one or more member participants 104. The use of the term “member” indicates subscription to the performance tracking system 100 by an individual, institution, or other entity, although non-subscribers may also interact with limited access to the performance tracking system 100. Although shown as single entities, it will be understood that a plurality of member participants 104 and/or member institutions 102 can exist in and operate in cooperation with the performance tracking system 100. A participant (also known as an applicant) that is interested in becoming a member of the performance tracking system 100 can begin by opening a World Wide Web site (herein, simply web-site) provided for and operated by the central server and database facility 106. Membership in the performance tracking system 100 includes access to a web-based program to register into the performance tracking system 100. The performance tracking system 100 includes a practice regiment and other information to prepare for one or more certified tests that evaluate various physical performance criteria, whether athletic, vocational, etc.

The central server and database facility 106 includes functionality to compare peer group certified test results and serve as a communication center for the transfer of member participants' certified test results, demographic information, and academic preferences to selected institutions of the member participants' choice. A web-site provided by the central server and database facility 106 can explain the program and the processes needed to participate. If a participant decides to join as a member (and thus becomes a member participant 104) of the performance tracking system 100, payment of a membership fee(s), such as via secure transactions, is required. After payment of the membership fee, a member participant 104 is issued an individualized membership number, which is the identifier of the participant for all further interactions. The member participant 104 can receive electronic transmissions via a web-site (or other mechanisms of information transfer) of information and opportunities that are included in the membership program.

If the member participant 104 decides to attend a testing session at an authorized performance lab 108, the locations and the dates of the test sites may be found on a web-site provided by the central server and database facility 106. Registration and confirmation for a test can be conducted via a web-site.

A web-site provided by the central server and database facility 106 may serve as a coordination center of the performance tracking system 100. One or more databases of the central server and database facility 106 can be based on software that functions to collect, receive, manipulate, analyze, process, compare, and/or communicate data that is inputted through the web-site and other secure resources.

A member institution 102 may include an organization that has an interest in receiving data that has been released by the member participant 104. The member institution 102 may include an academic institution, occupational organization, and/or a government agency. Thus, an institution can pay for a subscription (to become a member institution 102) to participate in the performance tracking system 100 and be allowed to query one or more databases (provided by the central server and database facility 106) in search of candidates or applicants that fit specialized criteria that has been submitted in a proscribed format. The criteria can be analyzed by software of the central server and database facility 106 using data in the aforementioned database(s). One or more member participants 104 can automatically receive a communication from the central server and database facility 106 that a specific member institution 102 has requested information about a member participant 104 that possess some or all of the characteristics that have been recorded in a collection of the data obtained from testing at a performance lab 108. The member institution 102 preferably does not receive any identifying reports on the member participant 104 that meets the criteria that the institution 102 has selected. Instead, the member participant 104 is given contact information for the member institution 102 that has expressed an interest, leaving it to the member participant 104 to contact the member institution 102.

There are preferably many performance labs 108 that are located, for example, on a geographical basis. The performance lab 108 is preferably authorized and licensed by administrators or authorized representatives of the performance tracking system 100. For an athletic performance-based performance tracking system 100, the performance lab 108 preferably conducts testing protocols that include but are not limited to those that measure body composition, endurance, speed/acceleration, muscle explosion/power, and agility/flexibility, although not limited as such. The performance lab 108 may be equipped with proprietary equipment and procedures used to conduct a testing program in a standardized manner with authorized, certified and trained evaluators. The performance lab 108 includes equipment that enables transmission of data to and from one or more databases of the central server and database facility 106.

FIG. 2A is a schematic diagram depicting one embodiment of a performance tracking system 100a. The performance tracking system 100a includes one or more of the following: a central server and database facility 106 and one or more performance labs 108. The performance tracking system 100a interacts with one or more member participants 104 and member institutions 102 over a medium, such as the Internet 210. The performance lab 108 includes one or more local area networks (LANs) 202 or other communication networks that supports a plurality of test stations, for example test stations (TS) 216a-c, which are served by one or more LAN servers or computers 205. The test stations 216a-c include test and measurement equipment and may or may not include test station modules (explained below) that receive and send information (e.g., data) to the test and measurement equipment as well as enable communication with the LAN server 205. When test station modules are not included, test and measurement equipment are directly coupled (cabled or wireless communication) to the LAN server 205. The LAN server 205 is coupled to the Internet 210, with or without an intermediary Internet Service Provider (not shown), as is true for other components shown. As is well known to those skilled in the art, the Internet 210 comprises and is coupled to a host of other networks (e.g., LANs, wide area networks, regional area networks, etc.) and users.

The central server and database facility 106 includes a central server 250 that is preferably provided with one or more central databases 230a, and is coupled to the Internet 210, among other networks not shown. Although the database 230a is shown as externally coupled to the central server 250, one skilled in the art would understand that the database 230a can be integrated into the central server 250 in some embodiments. The central server 250 includes performance tracking software (PTS) 252, which supports one or more LAN servers 205 of performance labs 108 that can be provided across many locales. The LAN server 205 can access the central server 250 via browser software, according to well-known mechanisms. Additional information on Internet-based communication and Web-interface generation that may be implemented in the performance tracking system 100a can be found in U.S. patent application Publication No. 2002/0,169,835 A1, published on Nov. 14, 2002, filed on Jan. 18, 2001, and herein incorporated by reference.

In one embodiment, the central database 230a can be maintained and updated, and licensed out for use by one or more users or facilities, such as a corporate or institutional research facility. Access to the central database 230a can be implemented over the Internet 210, or in other embodiments, a local copy can be maintained at the LAN server 205. In the latter embodiment, the LAN server 205 can support the test stations 216a-c, which, for example, may access the LAN server 205 via browser software at each workstation, according to well-known mechanisms.

Further, the mechanisms by which the test stations 216a-c access the LAN server 205 (or the LAN server 205 accesses the central server 250) include CGI (Common Gateway Interface), ASP (Application Service Provider), Java, among others.

One skilled in the art will also understand that the information of the database 230a can be stored on a digital video disc (DVD) or other storage medium. In embodiments where local copies are provided (e.g., local to the LAN server 205), the local databases can be run from the test stations 216a-c, network server 205, etc., and updated periodically or otherwise via the central server 250. Further, one skilled in the art would understand that communication among the various components of the performance tracking system 100a and with the member participants 104 and/or member institutions 102 can be provided using one or more of a plurality of transmission mediums (e.g., Ethernet, T1, hybrid fiber/coax, etc.) and protocols (e.g., via HTTP and/or FTP, etc.).

FIG. 2B is a block diagram of an embodiment of an example central server 250 that can implement the performance tracking software (PTS) 252. With continued reference to FIG. 2A, one skilled in the art will understand that functionality of the example central server 250 can be embodied in the test stations 216a-c and/or LAN server 205 (FIG. 2A), alone or in combination (i.e., in a single component, or distributed over several components), among other embodiments. Further, one skilled in the art will understand that additional components or different components with similar functionality can be included in the central server 250, and/or some components can be omitted, in other embodiments. The performance tracking software 252 can be implemented as an executable program, and may be executed by a special or general-purpose digital computer, such as a personal computer (PC; IBM-compatible, Apple-compatible, or otherwise), workstation, minicomputer, or mainframe computer.

The performance tracking software 252 includes a user-interface (UI) module 254, a statistics processing module 255, and a search engine 257, among other functionality to provide the various performance tracking system features. The user-interface module 254 provides display functions according to well-known underlying display generation and formatting mechanisms. The statistics processing module 255 provides for statistical processing of data, including median, mean, histogram, and/or descriptive statistics, among others, using well-known statistical processing mechanisms. Further, the statistics processing module 255 facilitates data processing integrity. For example, the statistics processing module 255 may detect (and thus alert administrators or others) mean or median shifts of a defined percentage, for example ±5%, on individual or group test data in light of existing cumulative data (e.g., nation-wide data, etc.), which may signal to administrators that the data is of suspect integrity. For example, such variations may signal to administrators that test equipment calibration (e.g., test stations 216a-216c, FIG. 2A) is inaccurate, the test equipment configuration is not set-up properly, and/or that there are equipment problems. From this information, administrators can query test officials, discard the data, and/or adjust the data according to defined specifications, among other actions. The search engine 257 provides database search methodologies according to mechanisms well-known in the art.

If implemented in hardware, as in an alternative embodiment, one or more of the functionality of the performance tracking software 252 can be implemented with any or a combination of the following technologies, which are all well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

Generally, in terms of hardware architecture, as shown in FIG. 2B, the central server 250 includes a processor 260, memory 258, and one or more input and/or output (I/O) devices 270 (or peripherals) that are communicatively coupled via a local interface 280. The local interface 280 can be, for example but not limited to, one or more buses or other wired or wireless connections, as is known in the art. The local interface 280 may have additional elements, which are omitted for simplicity, such as controllers, buffers (caches), drivers, repeaters, and receivers, to enable communications. Further, the local interface 280 may include address, control, and/or data connections to enable appropriate communications among the aforementioned components.

The processor 260 is a hardware device capable of executing software, particularly that stored in memory 258. The processor 260 can be any custom made or commercially available processor, a central processing unit (CPU), an auxiliary processor among several processors associated with the central server 250, a semiconductor based microprocessor (in the form of a microchip or chip set), a macroprocessor, or generally any device for executing software instructions.

Memory 258 can include any one or combination of volatile memory elements (e.g., random access memory (RAM, such as DRAM, SRAM, SDRAM, etc.)) and nonvolatile memory elements (e.g., ROM, hard drive, tape, CDROM, etc.). Moreover, memory 258 may incorporate electronic, magnetic, optical, and/or other types of storage media. Note that memory 258 can have a distributed architecture, where various components are situated remote from one another, but can be accessed by the processor 260.

The software in memory 258 may include one or more separate programs, each of which comprises an ordered listing of executable instructions for implementing logical functions. In the example of FIG. 2B, the software in the memory 258 includes the performance tracking software 252 and a suitable operating system (O/S) 256, such as WINDOWS, UNIX, LINUX, among other operating systems. The operating system 256 essentially controls the execution of other computer programs, such as the performance tracking software 252, and provides scheduling, input-output control, file and data management, memory management, and communication control and related services.

The performance tracking software 252 can be a source program, executable program (object code), script, and/or any other entity comprising a set of instructions to be performed. When a source program, then the program needs to be translated via a compiler, assembler, interpreter, or the like, which may or may not be included within memory 258, so as to operate properly in connection with the operating system 256.

Furthermore, the performance tracking software 252 can be written as (a) an object oriented programming language, which has classes of data and methods, or (b) a procedure programming language, which has routines, subroutines, and/or functions, for example but not limited to, C, C++, Pascal, Basic, Fortran, Cobol, Perl, Java, ASP, and Ada.

The I/O devices 270 may include input devices, for example but not limited to, a keyboard, mouse, scanner, microphone, etc. Furthermore, the I/O devices 270 may also include output devices, for example but not limited to, a printer, display, etc. Finally, the I/O devices 270 may further include devices that communicate both inputs and outputs, for instance but not limited to, a modulator/demodulator (modem; for accessing another device, system, or network), a radio frequency (RF) or other transceiver, a telephonic interface, a bridge, a router, etc.

The performance tracking software 252 also communicates with the database 230a via the local interface 280. As described above, the central database 230a can be external to or integral to the central server 250.

When the central server 250 is in operation, the processor 260 is configured to execute software stored within memory 258, to communicate data to and from memory 258, and to generally control operations of the central server 250 pursuant to the software.

The performance tracking software 252 and the operating system 256, in whole or in part, but typically the latter, are read by the processor 260, perhaps buffered within the processor 260, and then executed.

The performance tracking software 252 can be stored on any computer readable medium for use by or in connection with any computer related system or method. In the context of this document, a computer readable medium is an electronic, magnetic, optical, or other physical device or means that can contain or store a computer program for use by or in connection with a computer related system or method. The performance tracking system 252 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical). Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory.

FIGS. 2C-21 are flow diagrams that depict various performance tracking method embodiments provided by the performance tracking software 252 (FIG. 2B). The method steps (depicted in parentheses below) shown and described may occur in other orders, method steps may be omitted, and/or additional method steps may be added while being within the scope of the preferred embodiments of the disclosure. The various method embodiments illustrated in FIGS. 2C-2I will be described in cooperation with some exemplary user interface functional block diagrams, as illustrated in FIGS. 3A-3C. FIG. 3A is a web-site block diagram 300a that illustrates one embodiment of exemplary functionality provided by a web-site provided by the performance tracking software 252 (FIG. 2A) to enable an individual to interact with the performance tracking system 100 (FIG. 1). The blocks in FIG. 3A represent web-interface functionality. Although the blocks are shown interconnected, there is no particular process flow or control hierarchy implied by the interconnections, except as otherwise noted. The web-site block diagram 300a includes, in one embodiment, a user interface 302 which enables user interaction to functional modules that enable access to several categories of information, including benefits to joining 304, registration and edit functionality 306, and newsletter functionality 308. Under the benefits to joining 304, the user can access information about the benefits to being a member 310, the benefits to parents 312, institutions 314, and schools 316. Under the registration and edit functionality 306, the user can register as a member 318, an institution professional 320, a certified tester 322 (e.g., for the performance labs 108, FIG. 1), as well as edit his or her current member profile 324. Under the newsletter functionality 308, the user populates entries with contact information, including entries for email address 326, password, 328, first name 330, middle initial 332, and last name 334.

FIG. 2C is a flow diagram that depicts a method embodiment 252a to enable an individual to register to become a member participant 104 (FIG. 1). With continued reference to FIG. 3A, an individual who wants to become a member can enter the web-site provided by the performance tracking software 252 (FIG. 2A) via the Internet 210 (FIG. 2A) and register (e.g., such as by selecting an icon or entering text (not shown) corresponding to the register as a member functionality 318), including completing a parental consent form (201). Demographics, sports, and other information can be collected via questionnaires. The individual can then print out the parental consent form and payment form (e.g., if the individual is under-aged, the parental consent form may be required) (203). The parent of the individual preferably signs the form and selects a method of payment. The payment form and parent consent documents can be scanned and emailed to the central server and database facility 106 (FIG. 1) (or another location designated by the administrators of the performance tracking system 100, FIG. 1) or mailed to the same by the US Postal service or express carriers such as FedEx, UPS, or other carriers.

After receipt by the central server and database facility 106 (or otherwise by an administrator of the performance tracking system 100, FIG. 1) of the parental consent and payment, the database 230a (FIG. 2A) can be updated to record (and thus active membership) that the individual is now a member (205). Note that in some embodiments, payment and receipt of consent forms can be automated. An email can be sent to the individual (now a member participant 104, FIG. 1) notifying them that he or she is a member (207). The member participant 104 will preferably be given a temporary password (e.g., valid only for a limited time for security reasons or otherwise) for their initial sign-in. Upon first sign-in, the member participant 104 can be prompted to change his or her password. The member participant 104 can be presented with his or her membership certificate and have access to the member web-site, as represented functionally by the member web-interface block diagram 300b shown in FIG. 3B.

The web-interface block diagram 300b includes, in one embodiment, functionality for a member web user interface 336 that provides for calendar of testing 338, survey data maintenance 340, comparison of certified test results 342, and editing (as well as including viewing mailings and referring a friend options) 344. The web-interface block diagram 300b further includes functionality that provides such information as testimonials 346, trends 348, vendor product survey 350, and parental consent forms 352, as well as information on how to prepare for tests (not shown), among other items.

FIG. 2D is a flow diagram that depicts a method embodiment 252b to enable a member participant 104 (FIG. 1) to register for a test. An email can be sent to the member participant 104 informing him or her of an upcoming certified test and registration information (209). Such an email can be sent responsive to the member participant 104 registering for a test, or unsolicited (e.g., automatically) upon the database 230a (FIG. 2A) being updated with the member participant information (e.g., after parental consent received). Such test information may also be accessed from a web-site (e.g., see calendar of testing 338, FIG. 3B). The member participant 104 can then register for the test and email the registration (211). Alternatively, information on upcoming test dates and sites may be introduced with the initial entry form (e.g., step 203, FIG. 2C), in which case the individual can express an interest at that point as to what dates and times he or she would like to participate in testing. Responsive to receiving the parental consent and updating the database 230a, an email notification of the test date and site can be sent to the member participant 104. Information regarding test registration and information about one or more member participants 104 can be sent to a performance lab 108 (FIG. 1) (213). For example, the information can be downloaded to the test site LAN server 205 (FIG. 2A). The information can include a list of registered member participants 104 for the particular day, time, and location.

FIG. 2E is a flow diagram that depicts a method embodiment 252c to receive and process test data and inform the member participant 104 (FIG. 1) of the results.

Assuming testing has been performed, test information for the member participant 104 can be uploaded from the LAN server 205 (FIG. 2A) to the central database 230a (FIG. 2A) (215). Processing can be performed (217), including statistical processing, monitoring of data integrity (e.g., defined shifts in median or mean scores for a test group and/or individual), grouping of data, etc. The results or an indication that results are available can then be sent to the member participant 104 (219), or the member can access the results (e.g., without notice) shortly after taking the test and manipulate the format of the data to provide comparisons to peer groups, etc., such as by invoking the survey data maintenance functionality 340 (FIG. 3B) in a member participant web-site.

Each successive time, the member participant 104 (FIG. 1) can log-in to a web-site provided by the performance tracking software 252 (FIG. 2A) with a user ID and/or password. The member participant 104 can update his or her personal data as needed via the edit profile functionality 344 (FIG. 3B), and update any of his or her survey data as warranted via the survey data maintenance functionality 340 (FIG. 3B). The member participant 104 may sign up for scheduled certified tests either by selecting a specific site (e.g., performance lab 108, FIG. 1) or search for certified test sites (e.g., via calendar of testing functionality 338, FIG. 3B) that are convenient for them.

FIG. 2F is a flow diagram that depicts a method embodiment 252d to register an institution to become a member. An institution (i.e., an individual(s) representing the institution) preferably registers via the Internet 210 (FIG. 2A) to become a member institution 102 (FIG. 1) (221). A web-site may enable this functionality, as shown in FIG. 3A (register as an institutional professional, 320). An institution will have a background check completed for security reasons. Once the background check is complete, the institution (now a member institution 102) will preferably receive an email with their membership ID and temporary password (223). The member institution 102 may be prompted on first sign-in to change their temporary password.

Note that a similar procedure to that described in FIG. 2F for a member institution 102 (FIG. 1) may also be followed for a coach, athletic director, youth league director, or equivalent. For example, a youth league director undergoes a background check, which is completed for security reasons. The performance tracking software 252 (FIG. 2A) sends the youth league director an email with his or her membership ID and a temporary password. The youth league director is prompted on first sign-in to change their temporary password. The youth league director may be able to download a report on his or her student athletes that have parental consent to provide the test result data.

FIG. 3C is a member institution web-interface block diagram 300c that includes functionality for member institution access. The member institution web-interface block diagram 300c includes, in one embodiment, functionality for a web-interface 354, which enables editing of profiles and viewing of mailings 356, searching 358, provision of a contact results list 360 and vendor product surveys 362. The member institution 102 (FIG. 1) can update their personal data as needed, such as via the edit profile functionality 356.

FIG. 2G is a flow diagram that depicts a method embodiment 252e to enable a member institution 102 (FIG. 1) to search for member participants 104 (FIG. 1) who have criteria in which the member institution 102 has an interest. The performance tracking method 252e preferably provides search screens (e.g., via selection of the detailed search on members functionality 358, FIG. 3C) to the member institution 102 (225). The search screens enable a search on criteria such as demographic, test results, and/or interests. Responsive to receiving the criteria (227), a search is processed (229) (e.g., perform the search, manipulate data, etc.) and data is provided to the member institution 102 (231).

FIG. 2H is a flow diagram that depicts a method embodiment 252f to enable a member institution 102 (FIG. 1) to inform a member participant 104 (FIG. 1) of the fact that there is an interest in him or her by the member institution 102. The member institution 102 (FIG. 1) can review the search results from the search described in association with FIG. 2G. The search result may include a list of participant members 104 that match the search, as identified by an anonymous identification number accessed, for example, via the contact results list functionality 360 (FIG. 3C). If the member institution 102 wishes to contact a participant member 104, they will highlight the corresponding identification number. A member institution 102 can select multiple identification numbers corresponding to multiple member participants 104 on a given search. After selecting one or more identification numbers, the member institution 102 can send in the request (e.g., via email) (233). The performance tracking method 252f can automatically generate a standard email to the member participant(s) 104 informing the same of the interest by the member institution 102 (235). The email sent to the member participant 104 may include a uniform resource locator (URL) for the member institution 102, and/or contact information for the member institution 102.

When the member participant 104 (FIG. 1) receives the email, he or she can review the information and make the decision if he or she wishes to contact the institution. If so, the member participant 104 can directly contact the member institution 102 (FIG. 1) via the contact information provided by the member institution 102. Otherwise, the member can opt-out.

FIG. 21 is a flow diagram that depicts a method embodiment 252g to enable a test administrator to register to apply to become a certified test administrator (CTA) (for the performance lab 108, FIG. 1). A test administrator may access the web-site to register (e.g., register as a certified tester functionality 322, FIG. 3A). A certified test administrator accesses a web-site provided by the performance testing method 252g to obtain registration information (237), which is then responsively provided through the web-site (239). The registration information may include information about courses to take to become certified, and even an on-line course may be provided in some embodiments as administered through by the performance testing software 252. The CTA preferably undergoes a background check completed for security reasons. Once the CTA satisfactorily completes the course, and the background check is approved, the CTA is notified (e.g., via an email) with their membership ID and temporary password (241). The CTA may be prompted on first sign-in to change their temporary password. The CTA can update their personal data as needed. The CTA can download marketing information to advertise certified test dates and locations. The CTA can also schedule certified test sessions for posting on a web-site provided by the performance tracking system 252 (FIG. 2A).

FIGS. 4A and 4B are front elevation and side view schematics, respectively, of an embodiment of a test station module (TSM) 400. One or more test station modules 400 can be disposed at one or more test stations (e.g., test stations 116a-c, FIG. 2A) to communicate information to and from a LAN server 205 located at a performance lab 108, as well as to communicate information to and from test and measurement equipment located at each test station. The test station module 400 includes a handheld module 402 removably disposed in a cradle 408. The handheld module 402 includes a display screen 404 that can be a liquid crystal display (LCD), among others. The display screen 404 provides a mechanism to display the status (e.g., on/off, ready, error prompts) of the test station module 400. The display screen 404 also displays the member participants identification number, test process information, a menu, and messages and/or instructions to a test administrator (e.g., certified test administrator, or CTA). The handheld module 402 also includes a keypad 406 that enables configuration of the test station 116a-1 for a desired test purpose, and includes functionality to start a performance test, monitor performance, and accept results. In other words, the handheld module 402 enables a test administrator to administer a performance test. The test station module 400 includes a barcode scanner 410 to enable scanning of a predetermined identification number of a member participant 104 (FIG. 1). The test station module 400 also includes a banner 412 that can be comprised of practically any material, including vinyl. The banner 412 can be customizable to enable identification of a particular test station (e.g., agility test station). Further included are supports 420 on which the handheld module 402, barcode scanner 410, and banner 412 are supported. In addition, the test station module 400 also includes a light curtain 414 for use in cooperation with electronic/optical test and/or measurement equipment and an embodiment of a controller 416. The controller 416 includes an antenna that enables radio frequency (RF) communication with other devices, such as test and measurement equipment used for testing physical performance.

FIGS. 4C and 4D are front elevation and side view schematics that depict the controller 416 of FIGS. 4A and 4B. The controller 416 includes a display 422 (e.g., light-emitting diode, LED, CD, etc.), key pad 424, and a handle 426. The controller 416 also includes I/O ports 428 for communication with the LAN server 205 (FIG. 2A) and/or with the various test and/or measurement equipment.

FIG. 4E is a flow diagram that depicts a method embodiment 400a of the test station module 400 shown in FIGS. 4A-4B. The overall control of the test station module 400 resides at the controller 416, and thus the program sequence implied by the method steps are executed by logic of the controller 416. With continued reference to FIGS. 4A-4D, from a ready or activated status (401), the test station module 400 receives the member participant's ID number from the barcode scanner 410 (403). The ID is preferably encoded in the form of a barcode and worn by the member participant 104 (FIG. 1), such as around the member participant's wrist.

Such information can be provided over a cable or wire, or transmitted over air, such as via RF communication. The display 404 of the handheld module 402 presents the appropriate ID number for the member participant 104 (FIG. 1), as well as presents a message alerting the administrator that the test station module 400 is ready to proceed (405). Thus, after the member participant's ID is scanned and authenticated, the CTA may command the member participant 104 to assume the proper starting position for the particular test. Different tests have different starting position requirements for the member participant 104. For example, in an agility test, the member participant 104 may be required to place one of his/her hands at the starting line such that a “break” of an optical beam path is detected (e.g., by a sensor). As another example, flexibility and upper body strength tests may require the participant to sit on or against a template (e.g., such that the legs are positioned at a 30-degree angle and the shoulders/lower back are fully pressed against a wall). Once the CTA is satisfied that that proper starting requirements are met, he/she will press a key on the handheld module keypad 424. Thus, the test station module 400 waits for signals corresponding to selection of one or more of the keys of the keypad 406 (407), and continually waits until an input was received (409). If input was received, the test station module 400 responsively effects an audible sound (e.g., a “beep”) (411). The “beep” signifies to the member participant 104 that he or she is to start the test (e.g., to start running, bending, throwing, etc.). In some embodiments, other forms of alerting the test administrator can be used, such as tactile or visual alarms.

The controller 416 receives and analyzes measurement data and effects the display of the same in the display 404 of the handheld module 402 (413). The handheld module 402 prompts a message on the display 404 to determine whether the results are acceptable (415). If not, operation proceeds to step 405. Otherwise, the handheld module 402 prompts another query to determine whether there is a need or desire for a second opportunity to take the test (417). Test administrators preferably have the ability to reset the test for cause (e.g., someone trips, etc). If so, operation proceeds to step 405, otherwise the controller 416 stores the results in memory and transmits the results to the LAN server 205 (FIG. 2A) preferably when the controller 416 is idle (419), although other times of transmission may be implemented.

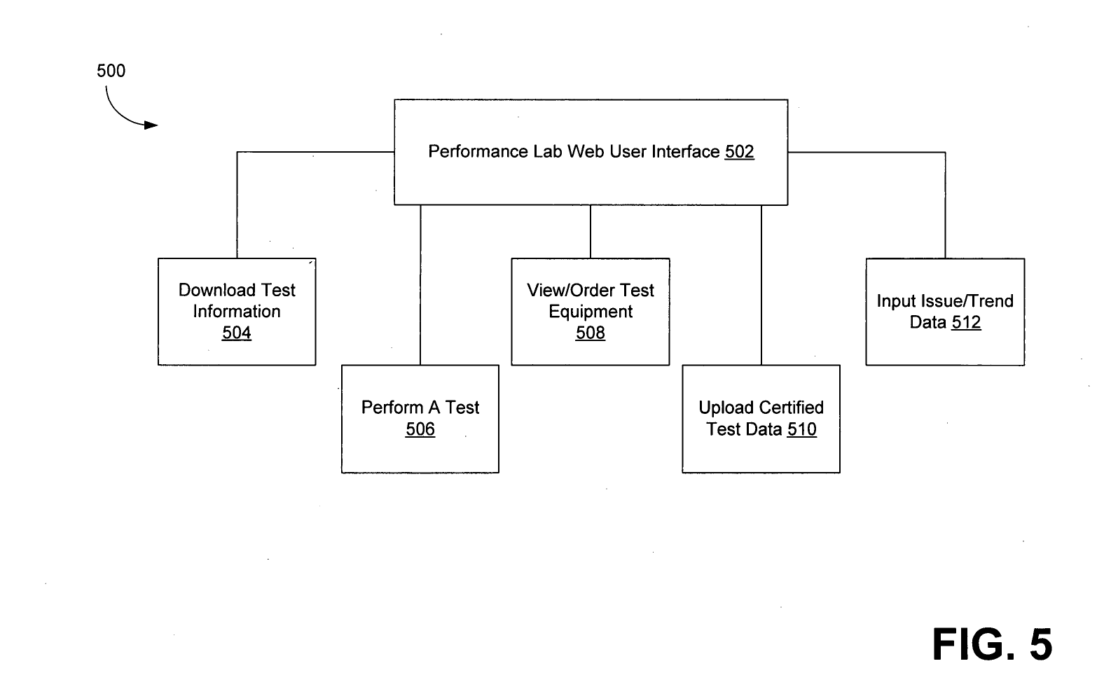

The LAN server 205 (FIG. 2A) communicates the data to the performance tracking software 252 (FIG. 2A) of the central server 250 (FIG. 2A). In one embodiment, the test administrator (or representative thereof), through the use of browser software at the LAN server 205, prompts a web-site provided by the performance tracking software 252 to enable the upload of the certified test data. FIG. 5 is a web-interface block diagram 500 that illustrates functionality for a web-interface embodiment provided by the performance testing software 252. In some embodiments, a user interface with like functionality may be provided by the LAN server 205. As shown, the web-interface block diagram 500 includes functionality for a tester web user interface 502, which enables downloading of test information 504, performing a test 506, viewing and/or ordering equipment 508, uploading certified test data 510, and providing issue/trend data 512.

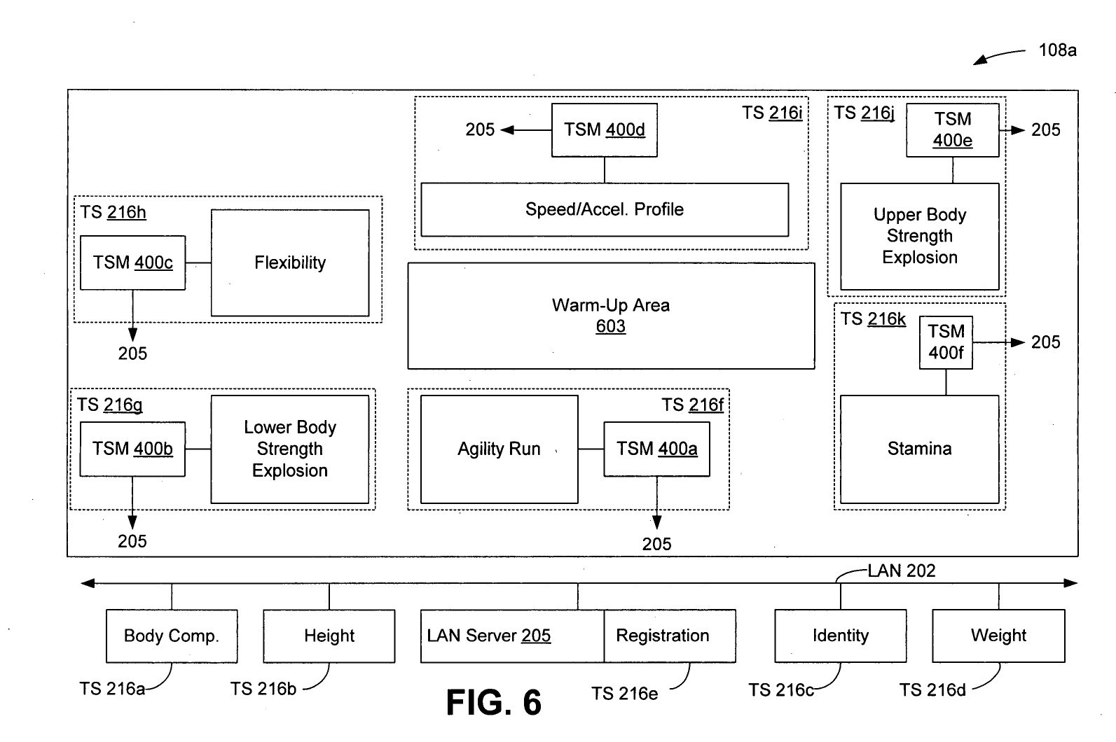

FIG. 6 is a schematic diagram that depicts one embodiment of a performance lab 108a. The performance lab 108a includes test stations 216a (body composition), 216b (height), 216c (identity), 216d (weight), 216e (registration), 216f (agility run), 216g (lower body strength explosion), 216h (flexibility), 216i (speed/acceleration profile), 216j (upper body strength explosion), and 216k (stamina). The test stations 216f-216k may surround a warm-up area 603. Test stations 216f-216k also include test station modules 400a-400f, respectively. The test station modules 400a-400f are coupled to a local area network 202, as are the test stations 216a-216e, which enables communication to and from the LAN server 205. Preferably, the equipment used in the test stations 216a-216k are constructed and operate to a defined standard, which can accelerate testing time and enable the systematic collection of data. In one embodiment, durability, serviceability, and ensured result integrity are important to the design and operation of the equipment, as well as designs that allow for intuitive and efficient operation in a tamper and foolproof package. The equipment is also preferably durable, with the ability to be stored in cases when not in use.

Once registered in the database 230a (FIG. 2A) of the central server 250 (FIG.2A) for a certified performance lab 108a, the member participant 104 (FIG. 1) can arrive at the performance lab 108a and register. The member participant 104 preferably receives an identification media, such as a pre-printed wristband or other article that has the member participant's identification number (e.g., the identification number designated via the performance tracking software 252 during registration) encoded in a barcode on the wristband. That identification number is activated once scanned by a barcode scanner, such as barcode scanner 410 (FIG. 4A). In some embodiments, barcodes may be printed out using a barcode printer (not shown) on-site. To insure integrity, each member participant 104 can utilize the identification media to register at one or more of the test stations 216a-216k.

A certified test administrator at each performance lab 108a can download all registered member participants 104 (FIG. 1) prior to the certified testing session, complete the tests on all member participants 104, and upload test data and other information to the central database 230a (FIG. 2A) via the Internet 210 (FIG. 2A) after the completion of the test regiment. For example, when all member participants 104 have completed their test regiment, all data from each test station 216a-216k can be downloaded into the LAN server 205. After all station data is loaded, the LAN server 205 can be connected to the Internet 210 and all data can be uploaded to the database 230a of the central server 250 (FIG. 2A). Alternatively, data may be “leaked” to the central server 250 throughout testing or periodically during testing, among other mechanisms for data transfer.

In one example implementation, a member participant 104 registers and receives his or her identification media. The member participants 104 progresses through the individual test stations 216a-216k, with the test results being recorded at each station. After one or more tests are completed, the individual test results for each test station 216a-216k are communicated to the LAN server 205. Such communication can occur via a variety of mechanisms, including via a LAN, wireless communication, or a combination of both, among other well-known mechanisms. The results from each test station 216a-216k are compiled at the LAN server 205. Once one or more tests have been compiled at the LAN server 205, the certified test administrator can “upload” the data via the Internet 210 (FIG. 2A) to the central server 250 (FIG. 2A).

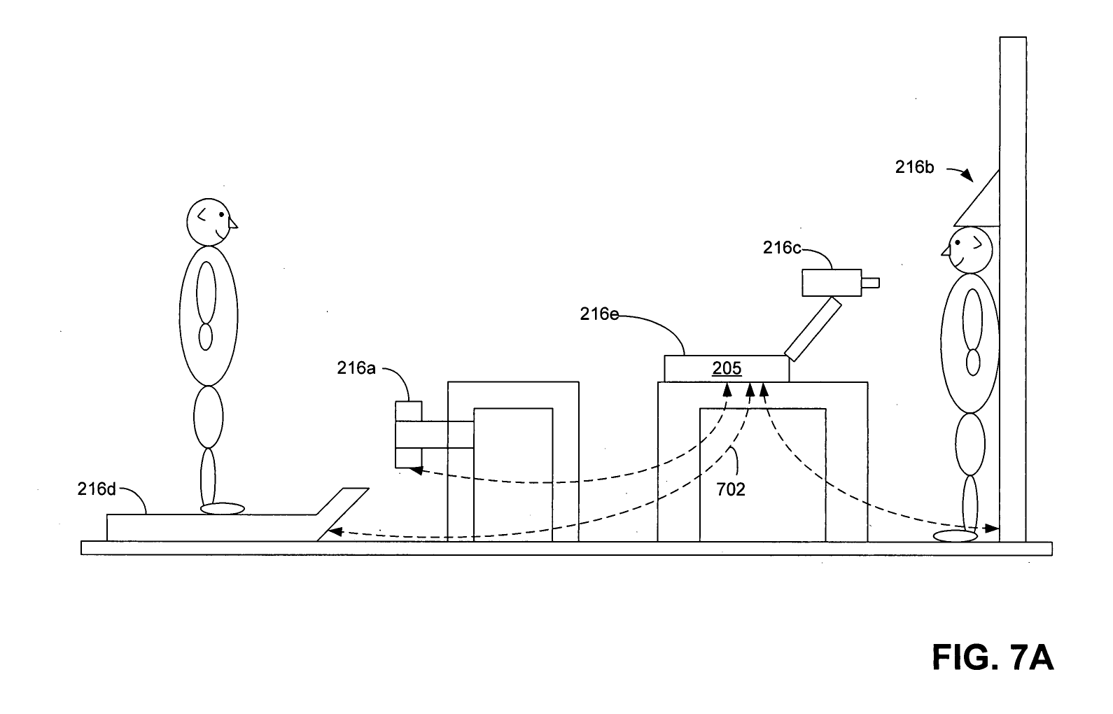

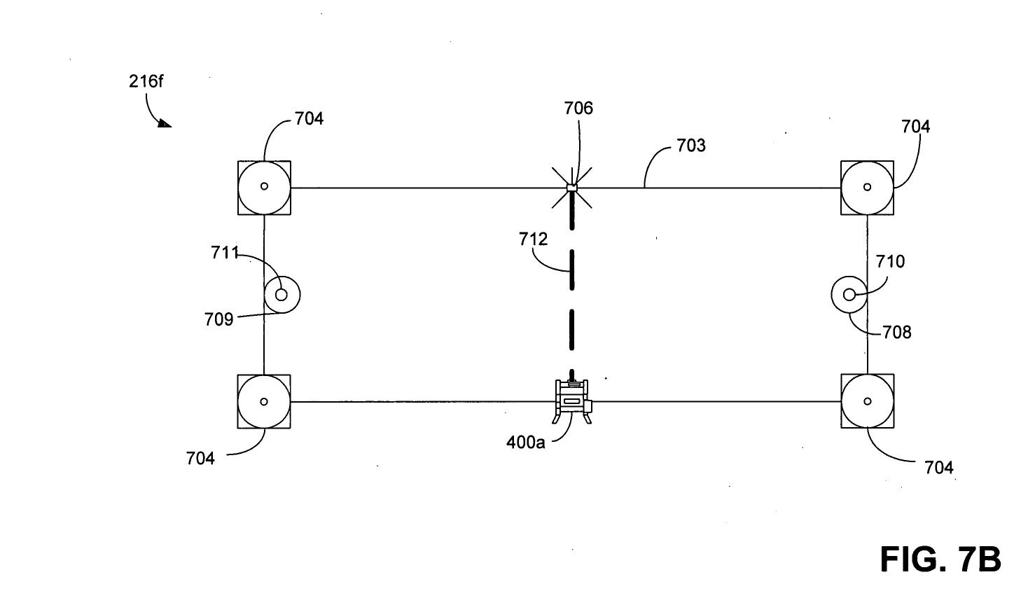

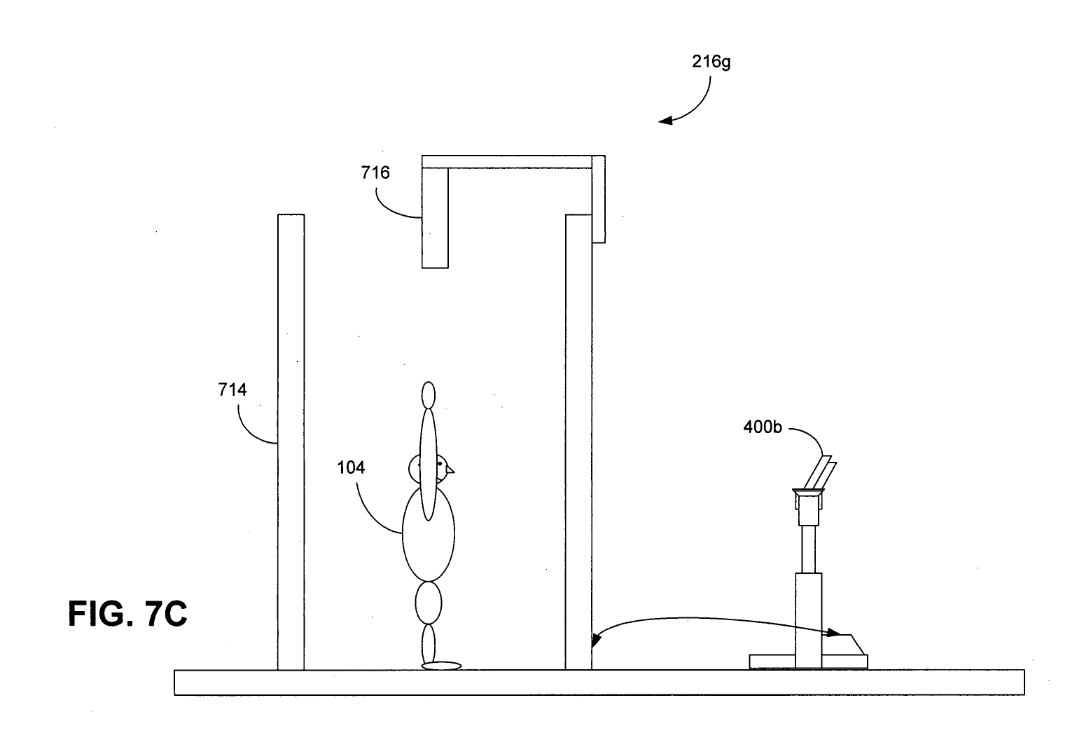

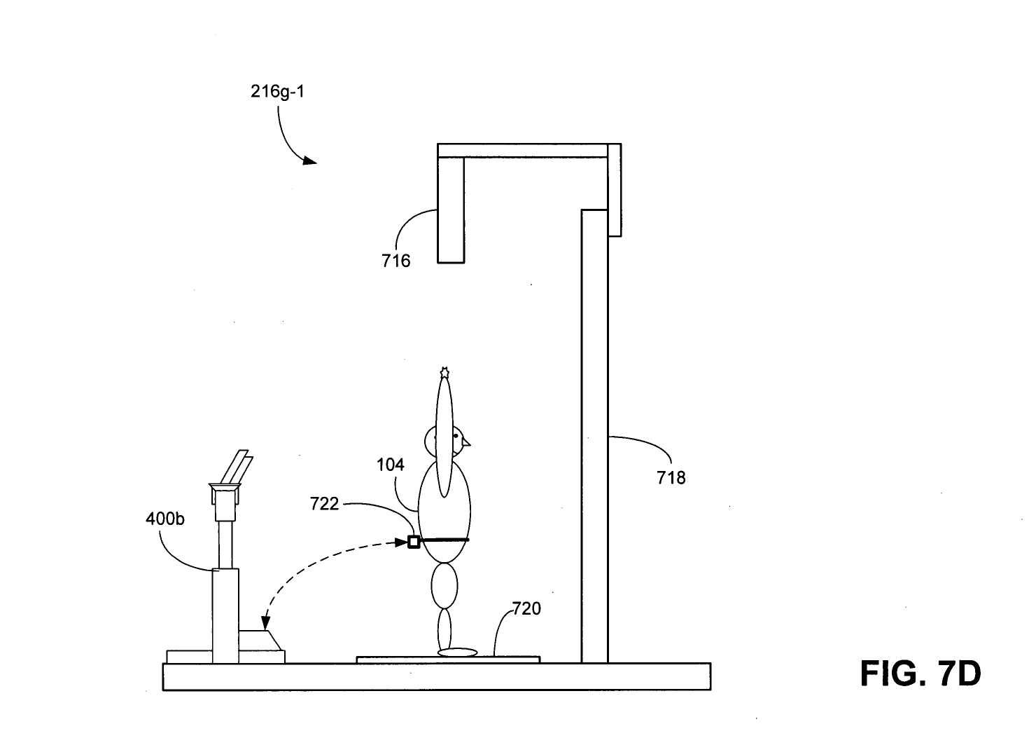

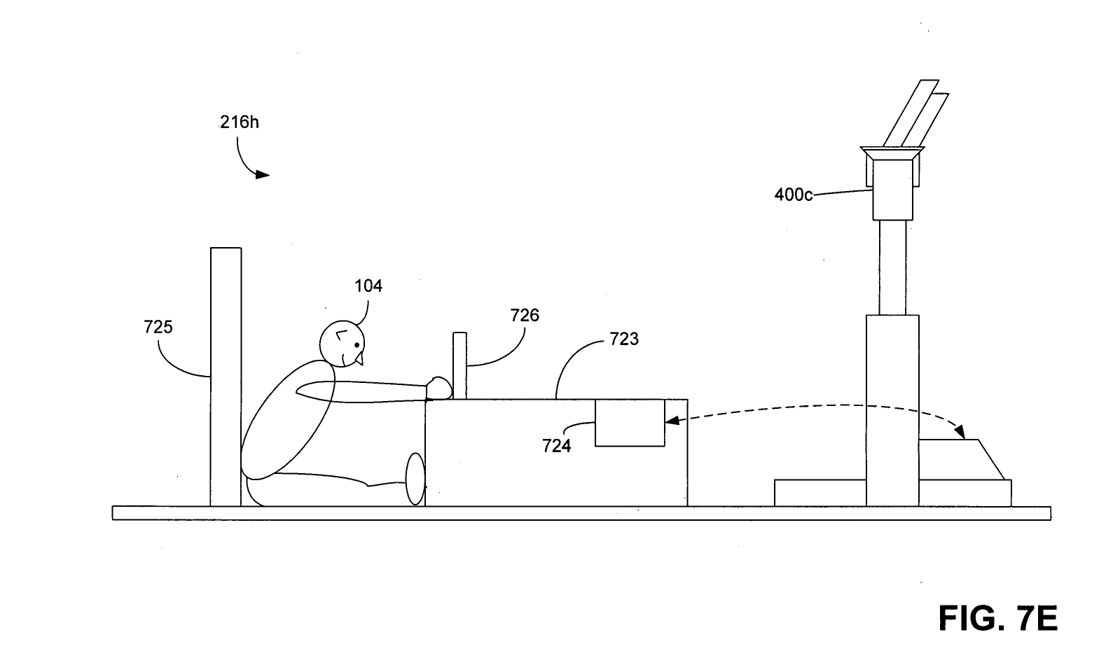

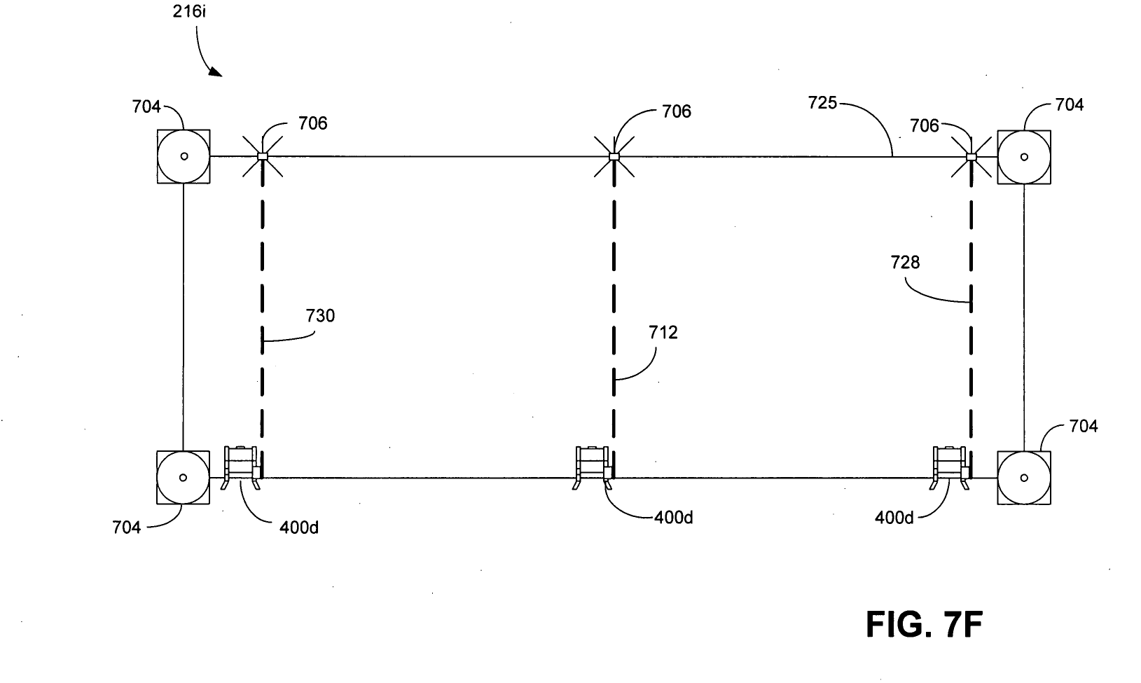

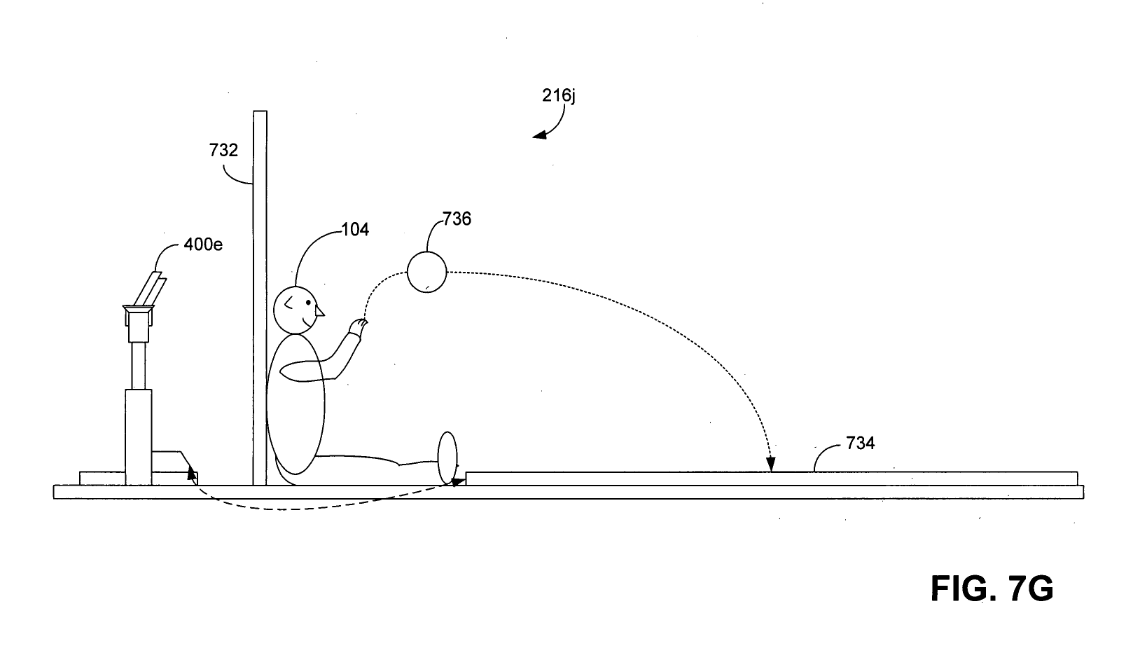

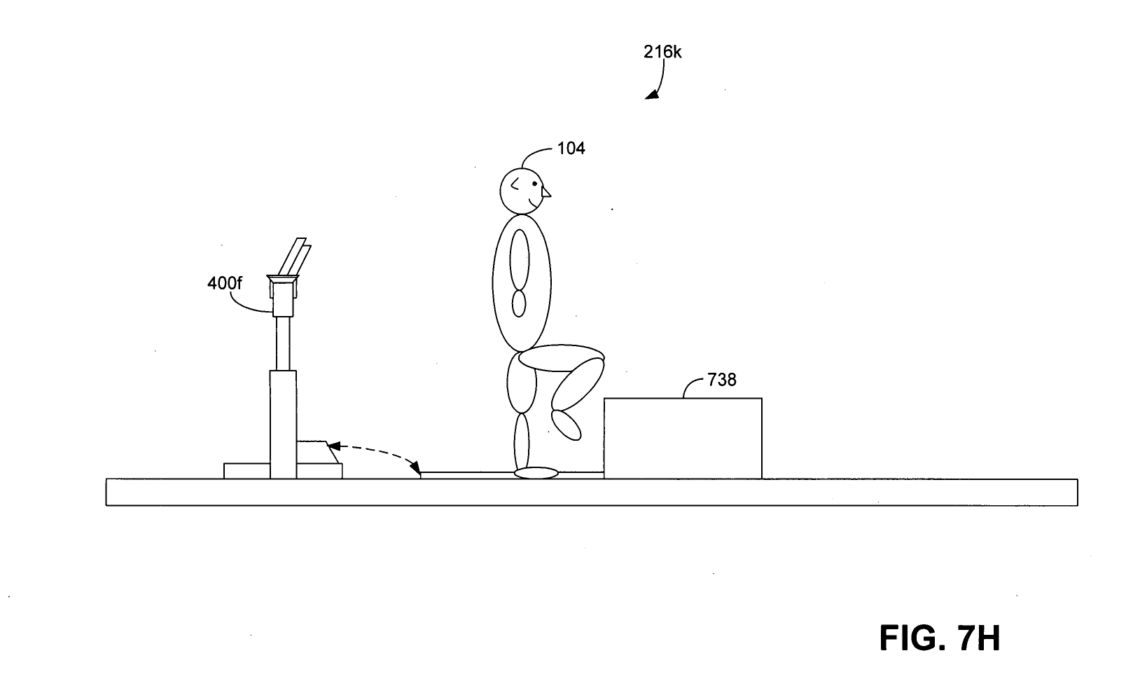

FIGS. 7A-7H are schematic diagrams that illustrate various test station embodiments as generally illustrated in FIG. 6. The performance lab 108a (FIG. 6) provides for physical tests that can be conducted during a student's certified physical performance test. Standardization of the testing regiment (e.g., types of tests, the manner of testing, etc.) is preferred, and as described above, equipment is preferably standardized to enable an acceptable (e.g., acceptable as determined by a recognized standards body or committee) degree of reproducibility and repeatability. A port is preferably provided on each test station module 400 (FIG. 4A) to add an optional digital display. An optional display may be used to show test results to others, such as the member participants 104 (FIG. 1), for instance to provide immediate feedback on results while reducing inquiries to each CTA, or to attract competitive interest. The equipment used in the performance lab 108a, if battery operated, preferably uses extended battery life technology and low battery indicators are preferably provided. All devices preferably have stable technology to eliminate or significantly reduce gymnasium interference (e.g., fluorescent lights, other test station devices, etc.). Alignment indicators are preferred to insure proper set-up (photo-electronic devices, etc.).

It will be understood that the example test stations 216a-216k and tests provided below are not meant to be limiting, and that some tests or test stations may be omitted, additional tests or test stations may be provided, or the described test stations or testing methods may be varied as would be understood in the context of this disclosure by those having ordinary skill in the art. Further, although digital devices are described throughout the disclosure, one skilled in the art would understand that analog technology can also be used, or a combination of digital and analog technology, and be considered within the scope of the preferred embodiments.

FIG. 7A is a schematic diagram that illustrates exemplary test stations 216a-216e. One or more of the test stations 216a-216e may be coupled directly to a LAN server 205, coupled to the LAN server 205 via the LAN 202, or integrated within the LAN server (e.g., registration test station, 216e). The dashed line with double-headed arrows (e.g., 702) represents communication, such as communication between test station 216d and the LAN server 205. Such communication may be enabled via cabling or via wireless technology (e.g., RF, infrared, etc.). Note that cabling is understood to include physical connectivity, such as via coax, hybrid/fiber, among others. Further, one or more of the test stations 216a-216e may be combined in a single device.

Test station 216a is a body composition apparatus, in one embodiment configured as a bioelectric impedance analyzer. The body mass index (BMI) and/or body fat percentage can be measured using the test station 216a or equivalent to determine each member participant's percentage body fat and BMI in relation to his or her age, gender, height, weight, and body build (e.g., youth, athlete, normal). Software in the LAN server 205 preferably automatically populates memory (not shown) in the test station 216a directly from previously recorded height and weight measurements (e.g., measured at test stations 216b and 216d, respectively), in addition to age, gender, and body build acquired from the downloaded registration data (downloaded to the LAN server 205 from the database 230a, FIG. 2A).

Test station 216b is a height measurement apparatus, in one embodiment configured as an electronic height measurement apparatus that includes a slidable disk that can be positioned to rest on a member participant's head and a height scale. A vertical measurement can be taken from the floor to the highest point on the member participant's head. The member participant 104 preferably faces directly ahead with arms by the sides. Shoes should be off, heels together, toes out at an approximately 45-degree angle and turned up with the weight on the heels. The test station 216b may include a foot pad with an outline of the feet pointed at approximately 45 degrees. The member participant's height can be measured to a minimum of approximately the nearest ¼ inch (which can be automatically translated to the metric system within the software of the LAN server 205 or the performance tracking software 252, FIG. 2A, or providing metric units and converts to English units). Other height measurement technology may include a bar code on a wall, laser, infrared, photocell, etc.

Test station 216c is an identity apparatus, in one embodiment configured as a digital camera.

Test station 216d is a weight determining apparatus, in one embodiment configured as a calibrated digital weight scale. Preferably, the member participant's shoes are removed and he or she should be wearing minimal clothing (shorts and T-shirt).

The test station 216d may include a digital readout scale (not shown) that can be used to obtain the member participant's weight to approximately the nearest one pound (which can be automatically translated to the metric system within the software of the LAN server 205 or the performance tracking software 252, FIG. 2A, or provided in metric units and converted to English units).

Test station 216e is a registration apparatus, in one embodiment configured as a software module in the LAN server 205 (although in some embodiments may be configured as a module that is separate from the LAN server 205). The test station 216e can be utilized by the certified test administrator to download those member participants 104 registered to take the certified test. The day of the test, the administrator can simply click and confirm the member participant 104. The member participant 104 can be given an identification media (e.g., using a plurality of methods including but not limited to bar code wrist band or similar technology) that can be used to identify the member participant 104 at each station.

FIG. 7B is a top-plan view of a test station 216f, which includes an agility run set-up in cooperation with a test station module 400a. Note that in some embodiments, the test station modules 400a-400f (for FIGS. 7B-7H) may be replaced, in whole or in part, with acquisition devices such as personal digital assistants (PDAs) or other devices configured with like functionality. The agility run set-up is configured with a running area 703 bordered on four corners with pylons 704 or other marking equipment. At opposite ends of the running area 703 are balls 710 (disposed on mat 708) and 711 (disposed on mat 709). Note that mats 708 and 709 may be rubber mats with a hole in middle for stabilizing the ball. The running area 703 includes a center-line 712, where a reflecting device 706 is located opposite to the test station module 400a. The agility run tests a member participant's ability to change direction laterally and accelerate while maintaining body control and balance. This ability is measured with running tests that require the member participant 104 (FIG. 1) to start, turn, and accelerate.

The agility test can be done using an electronically timed and recorded 20 yard shuttle run. The start from the center position (center-line 712) can be random as the member participant 104 may start to his or her right or left. The member participant 104 preferably places his or her hand on the floor breaking a starting line (center-line 712) that may be marked with an optical beam, or other marking mechanisms. After a specified delay, for example a two (2) second delay, an audible sound (e.g., from the test station module 400a) can let the member participant 104 know that he or she can start when ready. The timer can start when the member participant's hand leaves the starting field. The member participant 104 can break 5 yards, and pick up a ball 710 (e.g., a tennis ball) at mat 710, to register that they have completed a first leg. Then, the member participant 104 breaks back 10 yards crossing the center line 712, picks up a ball 711 at mat 709 to register they have completed the second leg 2. Then, the member participant 104 can run through the center line 712 recording the finish time.

The reflecting device 706 can be disposed, for example, approximately waist high (e.g., via 42-inch tripod mounts), and the test station module 400a can record the finish time. The time recording function inside the controller 416 (FIG. 4A) of the test station module 400a is preferably implemented with fast real-time timing circuitry to enable timing resolution, in one embodiment, to a thousandth of a second. Each participant may have, in one implementation, two opportunities on this test. Therefore, the software of the controller (e.g., controller 416) of the test station module 400a preferably stores two tests or has logic configured to pick the best score. If the controller stores two tests, the best score may be determined by the LAN server 205 (FIG. 2A).

The test station module 400a includes a light curtain 414 (FIG. 4A) for providing light to be reflected from the reflecting device 706, but other technology can be used as well, including a photocell, light emitter/detector module, etc., which can be mounted adjacent to the test station module 400a with or without the support of a mounting apparatus, such as a tripod. Some technology that can be incorporated into, or in cooperation with, the test station module 400a includes laser, infrared, touch pad, etc.

For example, the test station module 400a may use a photocell field or touchpad for starting.

The reflecting device 706 (described for this test and others) may be an optical reflector that reflects light transmitted from the test station module 400a. In some embodiments, recording functionality and/or light beam transmission functionality may be incorporated into a device disposed in place of the reflecting device 706, such that test data is transmitted from the device to the test station module 400a. Some features that may desired on such a device includes the provision for selecting one of multiple frequencies (e.g., 4 position dial and matching on receiving unit to pair transmission frequencies between transmitter and receiver).

The test station module 400a (and/or the reflecting device 706 or equivalent thereof) may also have additional features to improve test conditions, including electronic positioning to insure that equipment will not work without proper location (e.g., 10 yards apart, etc.) (or the provision for a template for proper set-up), minimum of approximately 0.5 mile range, and/or audible sound when field is broken or other alerting mechanisms. Other features may include, for starting position, allowing for the option of either a touchpad or photocell/infrared field, an audible sound incorporated with a 2 second delay when keying up for the start to activate start time (substantially eliminating “touch and go starts”), port to plug in an external stimulus start (light, horn, etc.), minimum RF interference, and capability of indoor or outdoor use.

FIG. 7C is a schematic diagram of a test station 216g, which includes a lower body strength explosion set-up in cooperation with the test station module 400b. The set-up includes two portable stands 714 (or a wall mount with like functionality), with one of the stands configured with an adjustable jump target 716. The test stands 714, in one implementation, may be a minimum of approximately 15 feet tall. The test station 216g is configured as a vertical jump/leg explosion test that can electronically measure and record jump height, using a photo cell field, by computing the difference between the member participant's standing reach and their vertical jump reach to the vertical target 716, thus enabling a vertical jump score. The test stands 714 may be coupled (wireless or cabled) to the test station module 400b. The test stands 714 include sensors or reflectors. Each member participant 104 may have, in one implementation, two opportunities on this test. Therefore, the software of the controller (e.g., controller 416) of the test station 400b preferably stores two tests or has logic configured to pick the best score. If the controller stores two tests, the best score may be determined by the LAN server 205 (FIG. 2A).

Note that in some embodiments, other vertical jump measurement technology may include laser, infrared, photocell field, among others. The member participant's jump distance is preferably measured continuous or a minimum to the ½ inch.

FIG. 7D is a schematic diagram of another test station 216g-1, which includes a lower body strength explosion set-up in cooperation with the test station module 400b. The set-up includes a single portable stand 718 (or wall mount with like functionality) with a jump target 716 attached thereto. The set-up also includes a pressure pad 720, which in some embodiments may be replaced with a photo cell field. An accelerometer 722 is attached to the member participant 104 for determining the vertical jump measurement (or providing data for the determination of vertical jump measurements), with communication between the accelerometer 722 and the test station module 400b occurring via wireless or cabling technology. Each member participant 104 may have, in one implementation, two opportunities on this test. Therefore, the software of the controller (e.g., controller 416) of the test station 400b preferably stores two tests or has logic configured to pick the best score. If the controller stores two tests, the best score may be determined by the LAN server 205 (FIG. 2A).

FIG. 7E is a schematic diagram of a test station 216h, which includes a flexibility test apparatus and a test station module 400c. The flexibility test apparatus is configured as a digital sit and reach box 723, having an adjustment handle 726 and a digital measurement scale 724 that is coupled to the test station module 400d. The digital sit and reach box 723 preferably has a scale (not shown), in inches or centimeters, on the box for immediate feedback to the member participant 104. The flexibility test can measure and score the core body flexibility (i.e., lower back, hips, and hamstring) using the electronic sit and reach box 723 or equivalent with an adjustment handle 726 for arm length. This test can measure each leg independently while the member participant 104 is seated. Although shown with the member participant 104 seated on the floor, in some embodiments, the member participant 104 may be seated on a bench. The flexibility test protocol may require that the member participant 104 must start with his/her lower back and the shoulder tightly against a wall 725. The member participant's flexibility can be measured to approximately the nearest ¼ inch (which can be automatically translated to the metric system within the software of the LAN server 205 (FIG. 2A) or performance testing software 252 (FIG. 2A), or providing in metric units and converting to English units). Other possible technology for measuring flexibility includes the use of bar code, laser, infrared, photocell field, etc.

FIG. 7F is a top plan view schematic diagram of a test station 216i, which includes an acceleration/speed profile set-up and one or more test station modules 400d. The acceleration/speed profile set-up is configured with a running area 725 bordered on four corners with pylons 704 or other marking equipment. Further included in the running area 725 is a center-line 712 and oppositely located end lines 728 and 730. Positioned at the center-line 712 and end lines 728 and 730 are test station modules 400d and measurement devices 706 (mounted, in one implementation, on 42 inch tripod mounts). The acceleration & speed profile testing can be accomplished by the member participant 104 (FIG. 1) running 20 yards (convertible to meters by the LAN server 205 (FIG. 2A) or the performance tracking software 252 (FIG. 2A), or conversion from metric to English). The time is measured electronically (e.g., measured in approximately 1000^{th} of a second) by the measurement devices 706 from the start (e.g., using a photocell or touchpad, not shown), for instance timed at the 10 yard point and 20 yard point. Therefore, there may be three measurements (2-10 yard split times and total time) for this test. This can enable the profiling of the member participant's ability to start, accelerate and finish a sprint. An option can be offered to the performance lab 108 (FIG. 1) to purchase an additional timing device (e.g., photocell) to measure a 40 yd sprint. This option may necessitate three split times for 10 yards, 20 yards, and 40 yards. The total time and three split times can be recorded. Each member participant 104 may have, in one implementation, two opportunities on this test. Therefore, the software of the controller (e.g., controller 416) of the test station 400b preferably stores two tests or has logic configured to pick the best score. If the controller stores two tests, the best score may be determined by the LAN server 205 (FIG. 2A).

Some embodiments may use other technologies for speed/acceleration measurements, including laser, infrared, photocell, etc. Wireless technology is preferred to eliminate the possibility of tripping hazard. Photocell or touchpad may be used for starting (preferably, a photocell).

FIG. 7G is a schematic diagram of a test station 216j that includes an upper body strength explosion set-up and a test station module 400e. The set-up includes a wall 732 against which the member participant 104 rests and a horizontal electronic field 734 that may be implemented using horizontal laser, infrared, or photocell field technology. The horizontal electronic field 734, in one embodiment, has a minimum distance capability of approximately 20 feet, and may be configured with a floor measurement chart template (not shown). The horizontal electronic field 734 is coupled to the test station module 400e. The upper body explosion and power test can isolate the upper body by placing the member participant 104 in a seated position with their back and hips against a wall. The distance the member participant 104 can throw a medicine ball 736 (e.g., using either a 16 lb. medicine ball or a 12 lb. medicine ball) is measured and recorded electronically. The member participant's legs can be positioned, in one embodiment, at an approximately 30 degree angle through the use of a template in one embodiment, not shown). Measurements can be from the wall (e.g., preferably measured continuous or minimum to the approximately ½ inch). Each member participant 104 may have, in one implementation, two opportunities on this test. Therefore, the software of the controller (e.g., controller 416) of the test station 400b preferably stores two tests or has logic configured to pick the best score. If the controller stores two tests, the best score may be determined by the LAN server 205 (FIG. 2A).

FIG. 7H is a schematic diagram of a test station 216k that includes a stamina set-up and a test station module 400f. The set-up includes a floor platform 738 (which may be a standard floor platform) configured with an optical or pressure sensor, and coupled to the test station module 400f. The member participant 104 steps from floor to platform and back. Technology could measure number of steps in a specific time period that can be programmed. The sensors measure (e.g., in approximately 1000^{th} of a second) the frequency the member participant 104 steps from floor to platform. This should be measured continuous. Other possible technology may include one yard or meter horizontal laser, infrared, photocell field, etc.

Any process descriptions or blocks in flow charts described herein should be understood as representing modules, segments, or portions of code which include one or more executable instructions for implementing specific logical functions or steps in the process, and alternate implementations are included within the scope of the preferred embodiments of the present disclosure in which functions may be executed out of order from that shown or discussed, including substantially concurrently or in reverse order, depending on the functionality involved, as would be understood by those reasonably skilled in the art.

It should be emphasized that the above-described embodiments of the performance tracking system 100 (FIG. 1), particularly, any “preferred” embodiments, are merely possible examples of implementations, merely set forth for a clear understanding of the principles of the performance tracking system 100. Many variations and modifications may be made to the above-described embodiment(s) without departing substantially from the principles of the disclosure. All such modifications and variations are intended to be included herein within the scope of this disclosure.

1. A physical test performance tracking method, comprising:
receiving standardized physical performance test data over a network from a test site, the standardized physical performance test data corresponding to physical performance for a plurality of individuals; and
processing the standardized physical performance test data to provide standardized data of physical performance among the plurality of individuals.

2. The method of claim 1, wherein receiving includes receiving from a plurality of test sites located at different geographic locations.

3. The method of claim 1, wherein the standardized physical performance test data includes data corresponding to occupational physical skills assessment.

4. The method of claim 1, wherein the standardized physical performance test data includes data corresponding to athletic physical skills assessment.

5. The method of claim 1, wherein processing includes providing at least one of a statistical numerical comparative score and a distinct set of numerical values.

6. The method of claim 1, wherein processing includes statistical processing.

7. The method of claim 1, further including providing access to the comparable data for an individual of the plurality of individuals to at least one of the individual and a third party.

8. The method of claim 7, wherein the third party includes at least one of an occupational institution and an academic institution.

9. The method of claim 1, further including informing at least one individual of the plurality of individuals that a third party is interested in contacting the individual based on the physical performance of the individual.

10. A physical test performance system, comprising:
a central database; and
a processor configured with first logic to receive standardized physical performance test data over a network, the standardized physical performance test data corresponding to physical performance for a plurality of individuals, the first logic configured to process the standardized physical performance test data to provide standardized data of physical performance among the plurality of individuals and store the standardized data in the central database.

11. The system of claim 10, wherein the processor is further configured with the first logic to receive from a plurality of test sites located at different geographic locations.

12. The system of claim 10, wherein the standardized physical performance test data includes data corresponding to at least one of occupational physical skills assessment and athletic physical skills assessment.

13. The system of claim 10, wherein the processor is further configured with the first logic to provide at least one of a statistical numerical comparative score and a distinct set of numerical values.

14. The system of claim 10, wherein the processor is further configured with the first logic to provide a web-interface that enables secured access to the comparable data for an individual of the plurality of individuals to at least one of the individual and a third party.

15. The system of claim 10, wherein the processor is further configured with the first logic to inform at least one individual of the plurality of individuals that a third party is interested in contacting the individual based on the physical performance of the individual.

16. The system of claim 10, further including a test site server, the test site server configured with second logic that receives the standardized physical performance test data from a plurality of testing stations and uploads the standardized physical performance test data to the central database in cooperation with the first logic.

17. The system of claim 16, wherein the second logic is configured on a computer readable medium.

18. The system of claim 10, wherein the first logic is configured on a computer readable medium.

19. A physical test performance tracking system, comprising:
means for receiving standardized physical performance test data over a network from a plurality of test sites separated geographically, the standardized physical performance test data corresponding to physical performance for a plurality of individuals; and
means for processing the standardized physical performance test data to provide standardized data of physical performance among the plurality of individuals.
